(19) United States
(12) Patent Application Publication
(10) Pub. No.: US 2007/0038181 A1
(43) Pub. Date: Feb. 15, 2007

(54) Method, system and device for delivering a substance to tissue

(76) Inventors: Alexander Melamud, Nesher (IL); Alexander Kalenkovich, Haifa (IL); Orit Shaked, Ramat-Yishay (IL); Ofer Nativ, Haifa (IL); Shay Kaplan, Givat Elah (IL); Yigal Natan Ringart, Givat-Elah (IL)

Correspondence Address: PEARL COHEN ZEDEK, LLP;PEARL COHEN ZEDEK LATZER, LLP, 1500 BROADWAY 12TH FLOOR, NEW YORK, NY, 10036, US

(21) Appl. No.: 11/499,878

(22) Filed: Aug. 7, 2006

Related U.S. Application Data
(60) Provisional application No. 60/706,435, filed on Aug. 9, 2005.
(60) Provisional application No. 60/772,376, filed on Feb. 10, 2006.

(51) Int. Cl. A61M 5/178; A61M 5/00; A61B 17/34; A61M 5/32

(52) U.S. Cl. 604/158; 604/159; 604/164.01; 604/173; 604/272; 606/186

(57) ABSTRACT

Devices and methods for delivering a substance to tissue or organs, particularly, the bladder, by a plurality of microneedles The devices may include a delivery tube, a a substance chamber to fill with the substance to be delivered, a plurality of needles, a plunger coupled to a handle movable relative to the tube to deliver the substance to the tissue through the needles, and a protective plate having at least one orifice therein, such that when the device is in a first, resting, position the needle tips are on a first side of the protective plate, and when the device is in a second, operational, position, the needles are on a second side of the protective plate.

### CROSS-REFERENCES TO RELATED APPLICATIONS

This application claims the benefit of U.S. Provisional Patent Application 60/706,435, filed Aug. 9, 2005 and U.S. Provisional Patent Application 60/772,376, filed Feb. 10, 2006 by the inventors of the present invention, the entire contents of both of which applications are hereby incorporated herein by reference.

### FIELD OF THE INVENTION

The present invention generally relates to the delivery of drugs, diagnostic agents or any other materials, compounds or substances into tissues or organs or other biological targets. More specifically, the invention relates to the delivery of substances to a target tissue or target organ by means of mechanical injection.

### BACKGROUND OF THE INVENTION

In treating disorders or diseases of the bladder (such as, but not limited to overactive bladder or bladder cancer tumors) there is a necessity to deliver drugs or other agents to the bladder wall at a specified location. Common practice for the delivery of drugs to the bladder wall, for bladder cancer treatment, is achieved through drug instillation, using a catheter inserted into the bladder, accompanied by a long incubation period. Other disorders are treated by a single endoscopic needle drug injection. These may be considered inefficient methods of drug delivery with relatively poor results and safety.

One way to deliver a drug into the bladder wall is by using a flexible, semi-rigid or rigid catheter, with a distal tip designed for dispensing drugs or other biological solutions or suspensions which have medical, biological, therapeutic or diagnostic applications for treating of the bladder wall. Such a catheter should be sufficiently rigid and long to pass through an endoscope-like device, such as a rigid or flexible cystoscope or resectoscope, used for the treatment of urinary tract diseases and disorders. The catheter-like device has a distal tip which, once positioned in proximity to the area that is to be treated, would rapidly and efficiently dispense or forcefully eject the drug(s) or other selected substance(s) in such a manner that the drug(s) or substance(s) applied as a solution, emulsion, suspension, or solid particles, would penetrate the tissue wall and remain lodged within the tissue.

Since the bladder wall is a thin tissue (typically, up to 10 mm thick), invasive treatments, such as an endoscopic needle pricking, can penetrate too deep and tear or perforate the bladder wall. On the other hand since many drug treatments are meant to penetrate only up to several hundred microns deep, it is difficult, using currently available methods and devices, to achieve such accuracy,

Treatment of large tissue areas can be done by multiple hypodermic injections. However, since the tissue integrity is damaged, much of the treating solution may clear from the tissue as the hypodermic injectors are withdrawn. Additional blood circulation and the elevated tissue pressure will hasten clearance of the remaining drug from the treated target area into the bladder Furthermore, physicians skilled in bladder treatment realize that directing a hypodermic needle several times in order to achieve a sufficient treatment area while ensuring penetration of the needle to a desired depth within the treated tissue requires great skill and manual dexterity.

In order to achieve prolonged therapeutic or diagnostic action, the therapeutic or diagnostic agents or substances or compounds or any other compositions may be packed in suitable slow-release capsules, as is known in the art. The capsules may also (optionally) include one or more colored substance(s) or dye(s) or pigment(s) for visually marking the treated area or region and for assisting the user to identify the regions of the prescribed target area to which the drug or substance has already been delivered. Such slow release capsules may remain in the tissue for a predetermined time and will degrade within the desired area or region realizing the desired substance or drug or composition slowly in a controlled manner

In case of drug instillation into the bladder, as for the treatment of superficial bladder cancer, dilution of the treatment solution, or substance(s) or suspension in the liquid contained within the bladder (or the other organ being treated) over time prevents drug's diffusion into the bladder wall.

Bladder cancer, which is the fourth most common cancer afflicting American men, accounts for more than 12,000 deaths annually in the US alone. Superficial bladder cancer (SBC) accounts for approximately 70% of all bladder cancer cases. Superficial tumors consist of papillary tumors confined to the mucosa, papillary or sessile tumors extending into the lamina propria, and carcinoma in situ, without muscle invasion. Most superficial tumors (60% to 70%) have a propensity for recurrence after TUR. Skilled urologists believe that TUR alone, though effective, is insufficient for treating all cases of superficial transitional cell carcinoma (TCC). Further and more intensive treatments are usually in the form of adjuvant intravesical immunotherapy or chemotherapy after TUR. Intravesical therapies, such as bacilli Calmette-Guerin (BCG) or Mitomycin C (MMC) are currently the most frequently used therapeutic agents. Most chemotherapeutic drugs, administered intravesically on a weekly basis, have not proved beneficial in preventing disease progression or mortality.

One shortcoming of immunotherapy or intravesical chemotherapy is the lack of sufficient continuous contact between the therapeutic agent and the cancer cells. The drugs, instilled into the bladder for approximately two-hour treatment period, may constantly be diluted by urine, resulting in poor penetration to the bladder wall. This phenomena may further be accelerated by the drug's physical properties, such as lipid solubility and molecular weight.

U.S. Pat. No. 4,524,770 discloses an add-on device, to be used in conjunction with either cystoscope or resectoscope, for injection of local anesthesia agents and occasional thermal treatment. Both functions are affected through a needle, by attachment to a long tube for the former and by attachment to electrical wires for the latter.

U.S. Pat. No. 6,730,061 discloses a needle injection device designed to inject large volumes of treatment solution into a tissue by means of several secondary hypodermic needles, enclosed within a larger primary needle, which extrude into the tissue once the distal tip is in place. The reference suggests, based on the secondary needle angle and length, that it is appropriate for use in large internal organs with substantial mass, such as the liver. The device disclosed would not be appropriate for use with thin tissue layers such as the skin or bladder epithelium, or for treating depths of tissue less than one millimeter.

U.S. Pat. No. 6,692,490 discloses a catheter-like device that is adapted for direct insertion, or in conjunction with an unspecified sheath, through the urethra for treatment of the genito-urinary tract. The disclosed device is described as predominantly having a barb or barbs for the conduction of electrical energies designed to be used for modification of tissue. The invention further discloses the possibility of being used for administering a wide variety of energy types including but not limited to, laser energy, radio frequency (RF) energy, microwave energy, infrared light (IR) energy, ultrasound energy and, combinations thereof. The reference describes that the disclosed catheter may be used for delivery of drug solutions to the distal tip of the catheter.

U.S. Pat. No. 6,689,103 discloses a device to deliver and inject fluid into heart tissue using an injection array. However, the device is not practical and does not solve problems of drug delivery and clearance.

### BRIEF DESCRIPTION OF THE FIGURES

The invention is herein described, by way of example only, with reference to the accompanying drawings, in which like components are designated by like reference numerals, wherein:

FIG. 1 is a schematic isometric diagram illustrating part of a needle array, usable in a multi-needle type catheter system, in accordance with an embodiment of the present invention;

FIG. 2 is a schematic part cross-sectional diagram of part of a catheter system including the needle array catheter of FIG. 1;

FIGS. 3A and 3B are schematic part cross-sectional diagrams illustrating two different operational states (before the injection procedure and during the injection procedure, respectively) of part of a multi-needle array catheter system, in accordance with an embodiment of the present invention;

FIG. 4 is a schematic part cross-sectional diagram illustrating a manually or automatically driven multi-needle catheter device useable in conjunction with an endoscope, in accordance with an embodiment of the present invention;

FIGS. 5 and 6 are schematic part cross-sectional diagrams illustrating two different stages in the operation of a self-cleaning multi-needle catheter system in accordance with an embodiment of the present invention;

FIG. 7 is a schematic diagram illustrating a region of tissue to be treated and the relative footprint of the area treatable by one treatment cycle of the catheter of FIG. 4;

FIGS. 8A and 8B are schematic part cross-sectional diagrams illustrating two different operational states (before the injection procedure and during the injection procedure, respectively) of a part of a protected multi-needle array catheter system, in accordance with an embodiment of the present invention;

FIG. 9 is a schematic cross-sectional diagram illustrating a manually or automatically driven multi-needle catheter device useable in conjunction with an endoscope, in accordance with an embodiment of the present invention;

FIGS. 10A and 10B are schematic cross-sectional diagrams illustrating two different parts of the multi-needle catheter system, in accordance with an embodiment of the present invention prior to the injection procedure;

FIGS. 11A and 11B are schematic cross-sectional diagrams illustrating two different parts of the multi-needle catheter system, in accordance with an embodiment of the present invention during the injection procedure;

FIGS. 12A and 12B are schematic cross-sectional diagrams illustrating two stages in the operation of the semi-flexible multi-needle catheter system in accordance with an embodiment of the present invention;

FIGS. 13A and 13B are schematic cross-sectional diagrams illustrating two different stages in the operation of the partially flexible multi-needle catheter system in accordance with an embodiment of the present invention;

FIG. 14 is a schematic cross-sectional diagram illustrating a manually or automatically driven injector with a multi-needle catheter useable in conjunction with an endoscope, in accordance with an embodiment of the present invention;

FIG. 15 is a schematic cross-sectional diagram illustrating a distal part of a multi-needle catheter system with an angled channel protector, in accordance with an embodiment of the present invention;

FIGS. 16A and 16B are schematic cross-sectional diagrams illustrating two different stages in the operation of the multi-needle catheter system with an angled-channel protector in accordance with embodiments of the present invention;

FIG. 17 is a schematic cross-sectional diagram illustrating the distal part of the dynamic multi-needle catheter system with an angled-channel protector, in accordance with an embodiment of the present invention;

FIG. 18 is a schematic cross-sectional diagram illustrating the multi-catheter delivery system, in accordance with an embodiment of the present invention;

FIG. 19 is a schematic cross-sectional diagram illustrating the distal part of the multi-catheter delivery system, in accordance with an embodiment of the present invention, and

FIG. 20 is a photomicrograph illustrating histology results taken from a C3H/eb mouse bladder tumor (MBT-2) after treatment with a multi-needle catheter embodiment of the present invention,

It is noted that all the drawing figures are schematic and the drawings are not drawn to scale or represent working plans for the assembly of the device therein described. The drawings should be referred to as schematic diagrams of the embodiments and are not intended to limit the scope of the patent or invention.

### DETAILED DESCRIPTION OF THE INVENTION

Devices, systems and methods of embodiments of the present invention provide for the delivery of solutions, emulsions, solids or suspensions into organs or tissue. The delivery may be performed through an intervening epithelium (or through any other intervening tissue layer) or directly into a tissue to be treated by using mechanical injection system to inject treating material(s), solution(s), suspension(s), emulsions, solids and the like, such that it may penetrate the tissue or organ to be treated, to a desired and effective penetration depth and at desired concentration.

The present invention may be used for delivery, into a tissue or an organ, of therapeutic or diagnostic agents in solution, emulsion or suspension form, including but not limited to, genes, proteins, drugs, pharmaceutical compositions, chemical compounds or drugs and mixtures thereof, natural and/or synthetic compounds (in an encapsulated or non-encapsulated form) or any other particulate form of useful diagnostic or therapeutic material(s), and/or substances and/or compounds and or compositions or mixtures thereof. The systems and devices of the present invention may deliver the therapeutic or diagnostic agents to a selected organ or tissue, such as, but not limited to the bladder, the prostate, the uterus, the heart or a chamber thereof (including but not limited to an atrium or the ventricle, the blood vessel and/or any other part of the vascular and cardiovascular system, the gastrointestinal tract, or parts thereof, the stomach or a part thereof, the bile duct, the gallbladder, the pancreatic duct, the pancreas, the stomach, the duodenum, the rectum, the sphincter, the esophagus, the colon, the small intestine, the genitor-urinary tract and/or parts thereof, the seminal vesicle, the uterus, the fallopian tube, the ovary, a testis, a sperm duct, organs having a lumen or any other desired internal organ of a human, a mammal or any other treatable organism, for the treatment of various organ disorders (such as, but not limited to bladder disorders, prostate disorders, various gastrointestinal disorders, disorders of the genitor-urinal tract or the like), including cancer growths, either alone or in conjunction with other surgical and/or therapeutic and/or diagnostic procedures in wide use for the treatment and/or diagnosis of such disorders.

It is noted that while the examples disclosed in the present application and described herein are mainly adapted for bladder treatment, the present invention is not intended to be limited to what is shown and described herein. Rather, the scope of the methods, devices and systems of the present invention is intended to include, inter alia, methods for treating any of various disorders of any organs and/or tissues and or body parts of mammals including but not limited to humans.

Furthermore, while the embodiments of the devices and systems of the present inventions are disclosed herein in a form adapted for use within different endoscopic devices (such as but not limited to resectoscopes, endoscopes, cytoscopes, enteroscopes, colonoscopes and the like), the devices and systems of present invention are not limited to the applications and forms disclosed. For example, the injection devices disclosed herein may be included within or adapted for being used within autonomous, self contained and/or robotic endoscopic devices such as but not limited to swallowable autonomous, free-moving and/or remote controlled gastrointestinal capsules and other autonomous endoscopic devices for use within a gastrointestinal tract or within the lumen of any other hollow organ.

Similarly, it will be appreciated by those skilled in the art that the injection and delivery devices of the present invention may be modified and adapted for use within any catheter system used for insertion into a blood vessel or into the lumen or internal space of any part of a cardiovascular system. Thus, the devices disclosed herein may be used, inter alia, for delivering a drug or substance or composition of substances in any of the forms described above into various parts organs or tissues of a cardio-vascular or vascular system with the same advantages described herein.

Such applications of modified devices may be used, inter alia, for delivering therapeutic or diagnostic substances and/or solutions, and/or diagnostic and/or therapeutic compositions, or the like to the wall or part of a wall of a blood vessel, to an aortic wall and parts thereof, an arterial wall and parts thereof, a venous wall or parts thereof the wall or parts thereof and to a cardiac chamber (including but not limited to an atrium wall or parts thereof, a ventricular wall or parts thereof, and the like).

The catheter system embodying the present invention may be inserted via a resectoscope or a cystoscope, or other endoscopic tool, into the bladder (or, possibly, other organs) and positioned for treating the bladder wall (or the other organ being treated) by pressing a distal tip thereof against the bladder epithelium (or the wall or surface or epithelium of the other tissue being treated), and injecting the substance thereinto.

Embodiments of the present invention allow for efficient drug delivery to the bladder wall (or to any other treated organ or tissue). Other possible advantageous aspects of embodiments of the present invention may be understood from the description of such embodiments below. For example, some embodiments of the invention may result in reduction in solution clearance, dilution and shortening of the overall treatment time. Furthermore, embodiments of the invention may enable finer control over depth of penetration of the therapeutic or diagnostic agent into the tissue (such as, but not limited to the bladder's epithelium), while reducing the risk of bladder wall perforation (or of damage to any other treated organs or tissues).

Needle injection for the implementation of drug delivery is typically described for trans-dermal delivery, without any invasiveness of device parts into the body of a human or animal. As will be further described below, and for the non-limiting exemplary application of drug delivery into the bladder in accordance with possible embodiments of the present invention, embodiments of the present invention may take into consideration design issues pertaining to catheter dimensions, for example, the length of the catheter, which may be long enough to pass through a resectoscope or cystoscope and reach the bladder wall; the catheter diameter, which may be thin enough to pass through the open device lumen of the resectoscope or cystoscope designed for the insertion of treatment devices into, and near, the afflicted site on the bladder wall.

The embodiments of the present invention may be designed to be able to provide sufficient doses of treating agent or substance to the bladder wall. Some embodiments of the invention may be designed for repeating the delivery of the agent, solution or substance to the bladder wall or other treated organ or tissue multiple times without requiring removal of the catheter between the multiple injections of the substance or agent.

Additionally, some embodiments of the device according to embodiments of the invention may be designed to be able to provide specific features to ensure the penetration depth of the needles, fixation of the needle position, an automatic retraction mechanism and prevention of occasional drug injection(s) using a valve system.

Embodiments of the invention may include a reusable handle with an automatic dispensing system capable of providing exact quantities of drug or any other agent for each injection, In accordance with an embodiment of the invention the handle may be designed to provide additional features, including, for example, a disposable catheter with a multi-needle head incorporated within the handle.

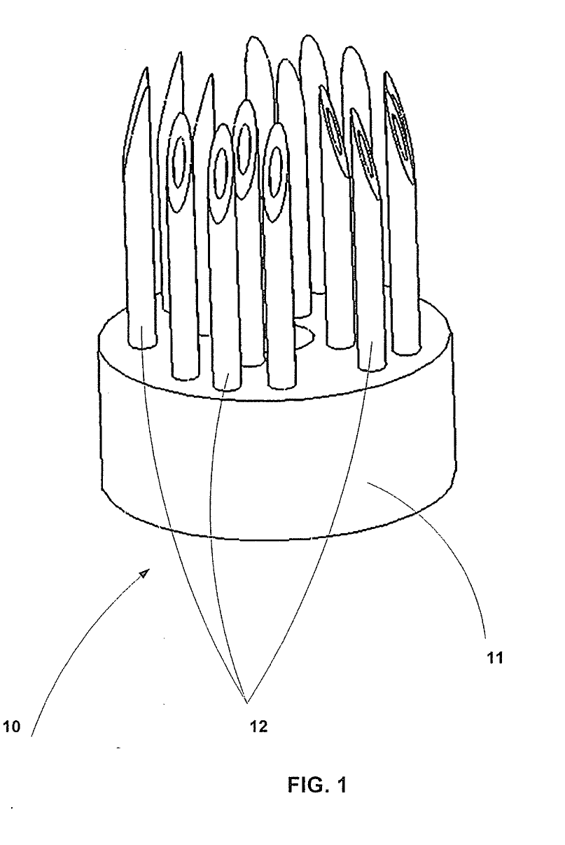

Reference is now made to FIG. 1 which is a schematic isometric diagram illustrating part of a needle array, usable in a multi-needle type catheter system, in accordance with the present invention. The needle array 10 includes a holder disc 11. At least one, but preferably multiple, hypodermic needles 12 are attached or affixed to or embedded in the holder disc 11 The needles 12 may either be identical or may differ from each other with respect any one, some or all of the following parameters: the needle length, needle angle, needle dispersal (e.g., variations in distance from a needle to other needles), the hypodermic shape, the direction of the tip and the tip diameter, Furthermore, the entire needle array 10 may have as few as one (a single) needle to as many as n needles with an effective outer cross-sectional area of one needle S_{needle}, in any variety of distributions to ensure optimal dispersion of the therapeutic or diagnostic solutions applied to the tissue through the needle(s).

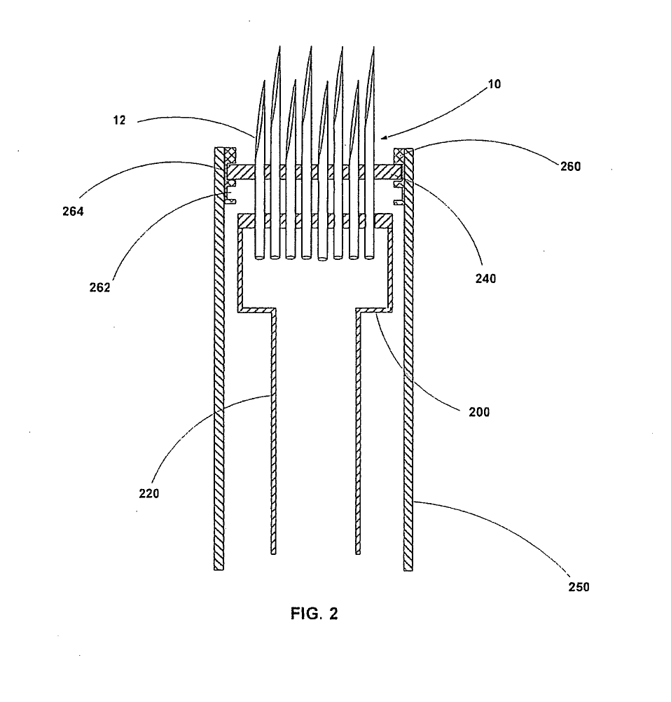

Reference is now made to FIG. 2, which is a schematic part cross-sectional diagram of part of a catheter system including the needle array 10 of FIG. 1. The needle array 10 may be attached within a needle holder 200 which is decreased or stepped down in diameter from the distal end towards the proximal end, forming a feed connector part 220 to which a suitable solution feed line (not shown) may be attached or connected. The delivery of the drug solution may occur after the needle array 10 is inserted into the target tissue to penetrate the tissue. In operation, the drug or the therapeutic or diagnostic solution may injected into the target tissue through a feeding line (not shown) and the needle(s) 12 of the needle array 10 by applying pressure using a suitable pump (not shown). The pump may be a peristaltic pump, a hypodermic pump or any other suitable pump, manual or automatic, that is configured to pump a solution or a liquid The needle array 10 and the needles 12 themselves may vary as described in the previous paragraph referring to FIG. 1.

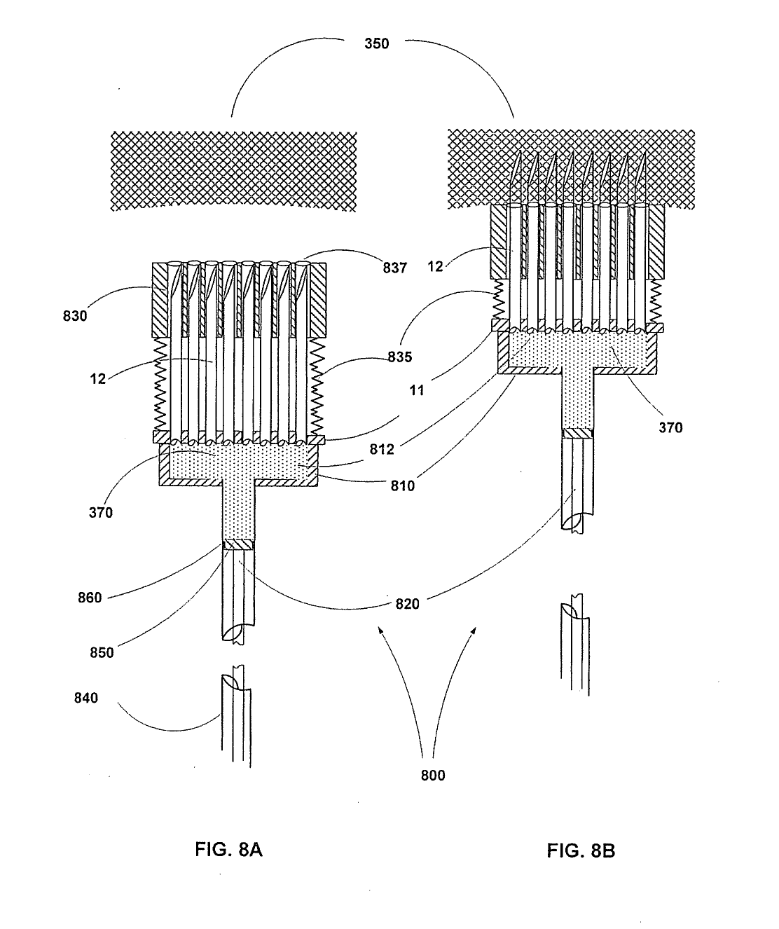

Furthermore, in accordance with an embodiment of the present invention, the needle working length (which determines the depth of penetration of the needles 12 into the target tissue) may be controlled by axially changing the location of an optionally adjustable protector-stopper disk 240. For example, the protector-stopper disk 240 may change its axial position within threaded tip 260, which in turn may be fixed to outer sheath tube 250. Varying a position of protector-stopper disk 240 from top location 264 to bottom location 262 of the threaded tip 260 allows changing the final working length of needles 12 and penetration depth. Adjustable protector-stopper disk 240 may be implemented in different ways within the scope of the invention. Examples include a sliding mechanism, a spring-loaded element with stoppers for different length, or any other suitable manually or automatically driven adjusting solution. The protector-stopper disc 240 may be a disc made, for example, of plastic material or stainless steel or from any other suitable medical grade material. The protector-stopper disc 240 may be perforated and may have holes formed therein to accommodate needles 12. The needles 12 may snugly fit into the holes formed in the stopper disc, so that the stopper disc may be longitudinally moved back and forth along the length of the needles 12. The depth of penetration of the needles 12 into the tissue may thus be controlled by suitably moving the protector-stopper disc 240 to a desired position along the length of the needles 12, thereby preventing insertion of the needles into the tissue further than the portion thereof protruding out of the protector-stopper disc 240. In some embodiments of the invention, the protector-stopper disc 240 may serve any or all of three functions, e.g., protecting the needles from being damaged during insertion of the delivery tube through the sheath and to the tissue, cleaning the needles from tissue debris, for example, upon retraction after injection in a reuse embodiment, and stopping the needles from proceeding beyond a desired depth into the tissue (see also, FIGS. 8A and 8B). It will be appreciated that the different functions may be served by different elements, for example, there may be one adjustable stopping element and a separate protecting element, etc.

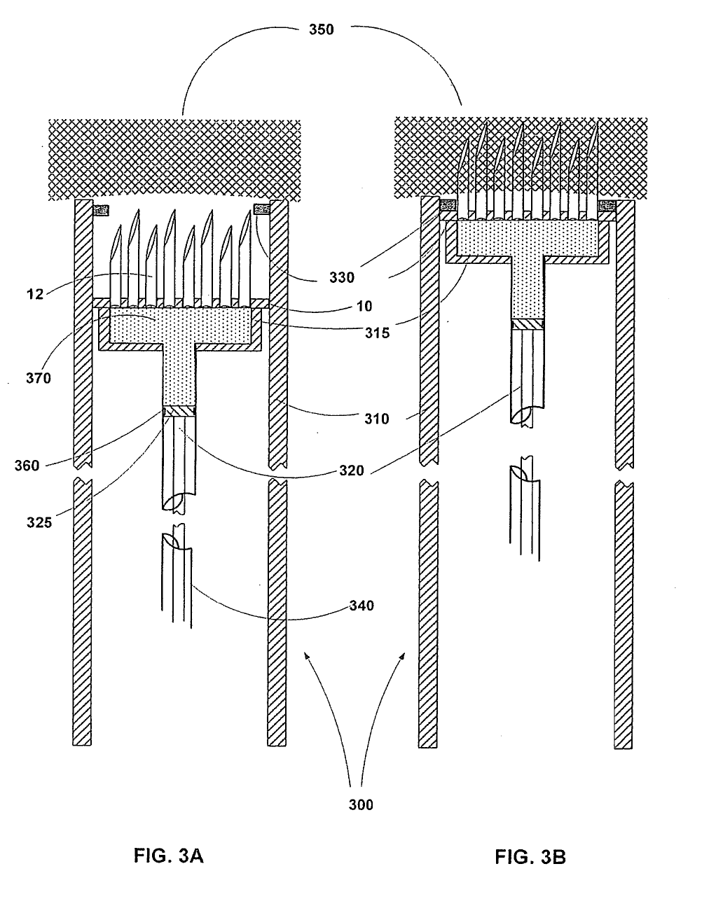

Reference is now made to FIGS. 3A and 3B which are schematic part cross-sectional diagrams illustrating two operational states (before the injection procedure and during the injection procedure, respectively) of part of a multi-needle array catheter system, in accordance with an embodiment of the present invention.

In FIG. 3A, part of a multi-needle catheter system 300 is illustrated. The catheter system 300 includes the needle array 10, a therapeutic or diagnostic agent solution chamber 315, a sheath tube 310, a plunger 320 and stopper pins or stopper disc 330. The distal part of the plunger 320 may include piston 325, which may be equipped with O-rings 360, which may fit into the circumference of the piston 325 and provide a leak-tight seal between the piston 325 and the walls of the delivery catheter 340.

In a first position, the distal end of the sheath tube 310 may be positioned adjacent to or in contact with the region to be treated on the surface of the bladder wall 350. The delivery catheter 340 may then be moved distally with relation to the sheath tube 310, until the advancement of the needles is stopped by stoppers 330, for example, by bringing the solution chamber 315 into contact with stoppers 330 and stopping the advancement of the chamber 315 thereby. Thus, in the second position, the needles 12 penetrate into the bladder wall 350 to a pre-defined depth limited by the stopper pins 330 (see FIG. 3B). By manually or automatically pushing the plunger 320 to a predetermined distance forward (in a direction distally or generally towards the needles 12) a certain volume of therapeutic or diagnostic agent solution 370 may be forced through the needles 12 and injected into the target tissue in the bladder wall 350.

After the delivery of therapeutic or diagnostic solution 370 to the target tissue 350, the catheter system is still in working position. Returning the catheter to the first position requires moving the delivery catheter 340 back to the proximal end of the sheath tube 310. Before the catheter system can be reused to deliver another dose or aliquot of therapeutic or diagnostic agent 370 to another part of the bladder wall 350, the sheath tube 310 may be positioned adjacent to or in contact with the new region to be treated on the surface of the bladder wall 350. The required volume of therapeutic or diagnostic agent solution 370 is filled into the chamber 315 and the process may be repeated. Each injection delivers a required equal portion of diagnostic agent solution 370 to the known effective surface of the target tissue 350. The required injected volume for each injection is defined by the type of the drug or other agents dissolved or suspended in the solution 370 and may be controlled by a dispensing system, which may be located, for example, at the proximal end of the needle catheter (not shown). The effective area for each injection may be characterized by the effective surface of the needle array 10. For instance, if a 27 F (1 French equals a ⅓ millimeter) resectoscope or cystoscope is used for the procedure, a space of only 15 F (4.5 millimeters) diameter may be used for inserting the needle catheter of the invention. Taking into account the ellipsoidal shape of the resectoscope, this allows the insertion of a catheter having a circular cross-section with a diameter of approximately 3 millimeters.

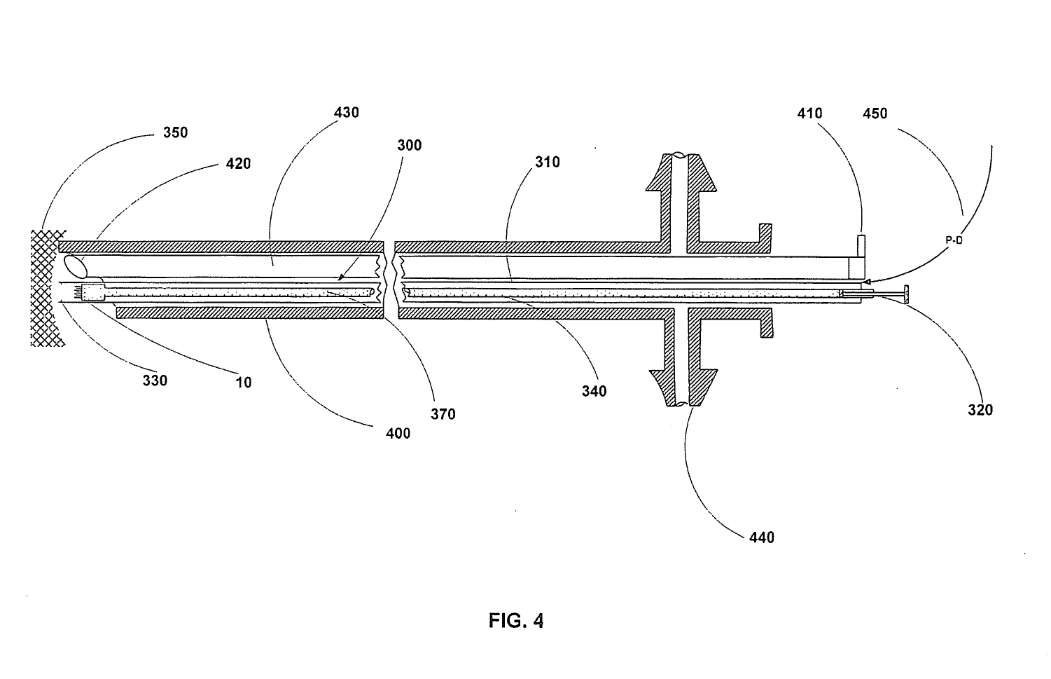

Reference is now made to FIG. 4 which is a schematic part cross-sectional diagram illustrating a manually or automatically driven multi-needle catheter device system 300 useable in conjunction with an endoscope, more specifically a resectoscope 400, for the injection of a therapeutic or diagnostic agent to the bladder wall 350. At the proximal end, the catheter device system 300 may be driven manually or automatically by a delivery plunger 320. A multi-needle array 10 is suitably attached or connected to the distal part of the delivery catheter 340. The therapeutic solution 370 fills the hollow space of the delivery catheter 340. The delivery catheter 340 is inserted into the sheath tube 310 which has stopper pins 330, limiting the penetration depth of the needles (located in the multi-needle array 10). At the proximal part of the endoscope 400 the multi-needle catheter device system 300 is fastened by fixation screws of the resectoscope adapter (not shown), which prevents accidental removal of the multi-needle catheter device 300 from the endoscope 400.

The endoscope 400 provides the means for observing, and guiding the catheter during the procedure via a monitor (not described) connected to an optical inlet 410 on the resectoscope. The optical lens 420 is attached at the distal tip of a telescope 430, facing the direction and the area affected by the catheter system. Two irrigation outlets 440 in the resectoscope provide the means to remove debris and/or liquid from the bladder during the TUR. There are also several possible ways of catheter device system 300 installations. One way is from the proximal part of the resectoscope via mechanical adapters (not shown in detail) to its distal part (P-D), and another way of installation is from the distal part to the proximal part of the resectoscope (D-P) via another type of mechanical adapters (not shown). The arrow 450 shows a (P-D) installation direction, The described multi-needle catheter device system 300 may be installed within the resectoscope in both directions (D-P and P-D).

Further to this embodiment, the application of therapeutic agent for the purpose of treating a bladder disorder is performed in conjunction, and immediately subsequent to TUR of a superficial cancer growth from the bladder wall. To cover a relatively large surface of the bladder, the treatment provider should be able to identify the tissue area already treated so as to provide optimal treatment coverage. The multi-needle catheter device system 300 may be rigid or flexible and may preferably be fully disposable. The proximal part of the catheter may be connected via a suitable connector to a standard medical syringe or to any other type of manual or automatic dispensing system, as is known in the art.

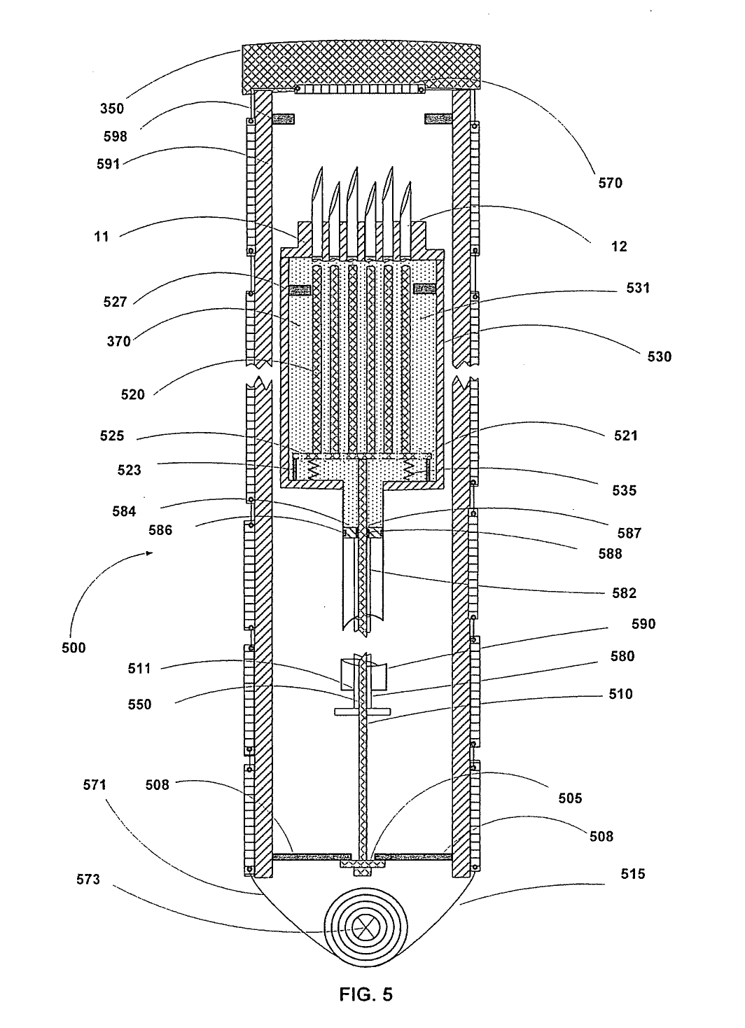

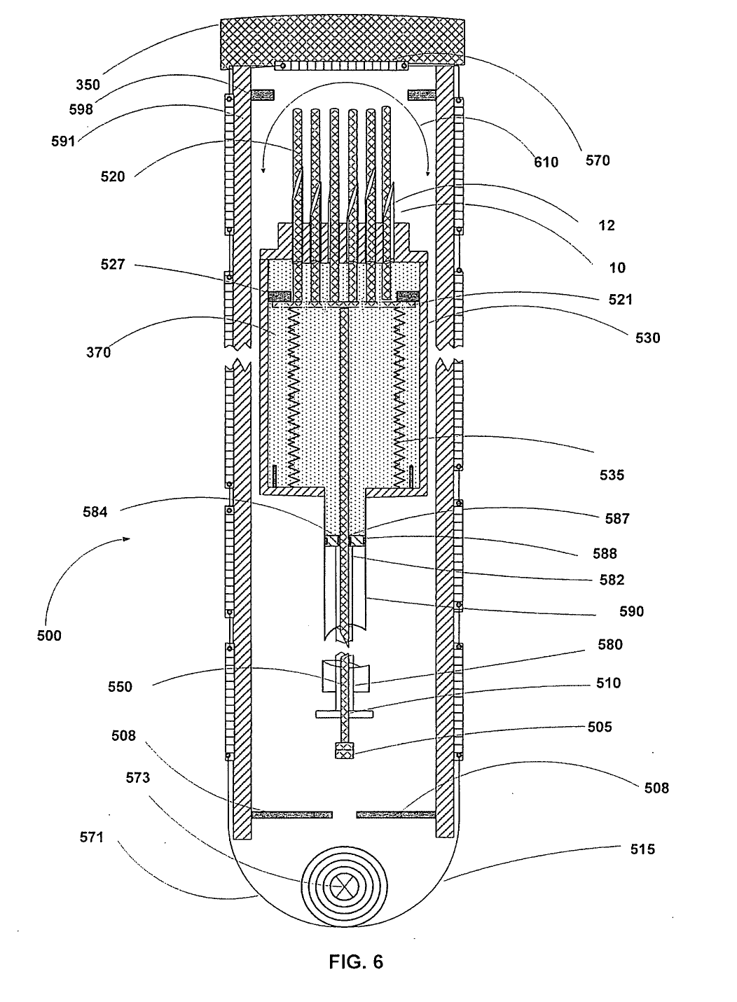

Reference is now made to FIGS. 5 and 6 which are a schematic part cross-sectional diagrams illustrating two different stages in the operation of a self-cleaning multi-needle catheter system in accordance with an embodiment of the present invention. The self-cleaning multi-needle catheter system 500 differs from the other types of needle based catheter systems disclosed hereinabove by including a needle cleaning system. The catheter system 500 includes an acceleration chamber 530 having a needle holder disk 11 sealingly attached thereto. The internal space 531 of the acceleration chamber 530 may be filled with the solution 370 of a drug and/or a therapeutic agent, and/or a diagnostic agent, as disclosed in detail hereinabove. The needle holder disk 11 is attached to the distal end of the chamber 530 and has hollow needles 12 suitably attached thereto, such that the solution 370 may be injected through the needles 12 when pressure is applied to the solution 370.

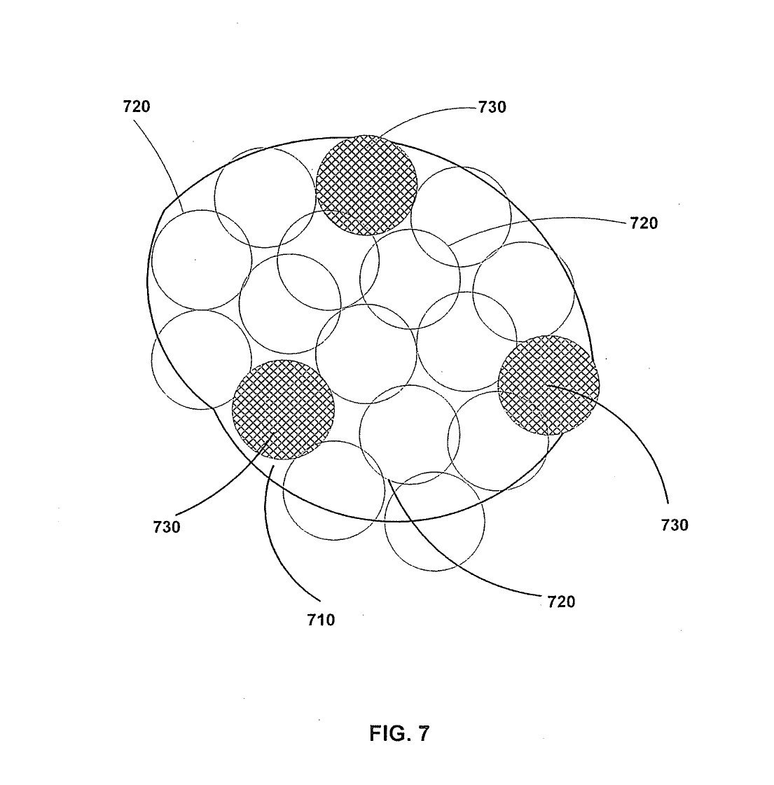

The needle cleaning system of the self cleaning catheters system 500 includes a cleaning plunger 510 and a cleaning membrane mechanism 515. The cleaning plunger 510 and the cleaning membrane mechanism 515 perform the cleaning of the inner bores and of the outer parts of the needles 12, respectively This cleaning procedure may be advantageous in the case of bladder cancer treatment where multi-injection procedure is required (i.e. multi bladder tissue penetrations with the needles 12 of the catheter system 500 at multiple tissue regions) in order to ensure proper agent or drug dispersal in the entire area or region to be treated (see FIG. 7 for the schematic representation of the multi repeated treatment for covering a large area needed to be treated). In the case of multiple injection sites the relocation of the catheter may result in re-insemination of cancerous cells into healthy areas. Such re-insemination of cancerous cells may be caused by the passive attachment of cancerous cells to the needles 12 during drug, therapeutic or diagnostic agent, or slow release capsule injection procedure. Such adhering cancerous cells may be transferred into a healthy bladder tissue region during the next catheter insertion and needle penetration into another tissue region.

The cleaning plunger 510 includes a cleaning plunger rod 550 which is attached to a piston 521. Cleaning pins 520 are attached to the piston 521 and are located and suitably aligned under the respective lumens of each of the needles 12. The number of the cleaning pins 520 is preferably equal to the number of the needles 12. When the cleaning plunger 510 is pushed forward (distally), each of the cleaning pins 520 enters and passes through the lumen of the corresponding needle 12, cleaning the inside bore of the lumen.

The needle cleaning system of the self cleaning catheters system 500 may also include the cleaning membrane mechanism 515 which includes membranes 570, attached to a movable strip 571 movably coupled to a rotating driving element 573. The Membranes 570 may be made of any biocompatible material, such as but not limited to Teflon®, silicon or any other suitable material.

The piston 521 may be perforated to contain orifices 525 to ease its movement within the therapeutic agent solution 370, stored in chamber 530. Springs 535 connects the piston 521 to the bottom part of the chamber 530. The injection plunger 580 includes an injection rod 582 which is connected to the injection piston 584. The piston 584 includes an o-ring 586 which fits into the circumference of the piston 584 and which provides a tight seal to avoid fluid leaking between the moving piston 584 and the walls of the delivery catheter 590. The moving piston 584 has a lumen 587 formed therein. The lumen 587 is sealed trough its circumference by an o-ring 588, which prevents the leakage of the therapeutic agent solution 370 and allows smooth movements of the cleaning plunger rod 550 within the injection rod 582.

The delivery catheter 590 is connected to the chamber 530 at its proximal part and includes an inlet port 511 for the cleaning and injection plungers 510 and 580, respectively at its distal end. In this way, the chamber 530, the plungers 510 and 580 and the needle holder disk 11 are effectively connected forming a functional unit. The delivery catheter 590 travels within the endoscopic sheath tube 591 while the strip 571 and the membranes 570 may be advanced or be made to slide or to be moved along the outer side of the sheath tube 591 by operating the rotating driving element 573 which is coupled thereto.

The piston 521 has two main positions within the chamber 530, a resting position in which the piston 521 is forced as far back as possible towards the proximal end of the chamber 530 by the cleaning plunger 510, while pressing the spring 535 which is limited by stoppers 523. This position is fixed by locking the cleaning plunger 510 using the pins 508 and the locking handle 505. In its working position, as best illustrated in FIG. 6, the piston 521 is pushed forward manually or automatically toward the distal end of the chamber 530. The forward movement of the piston 521 is limited by stoppers 527. At the working position, the cleaning plunger 510 is unlocked by turning the locking handle 505 by 90 degrees, as is best seen in FIG. 6.

At resting position, the multi-needle catheter system 500 is positioned adjacent to or in contact with the region of the bladder wall 350 to be treated, as is best seen in FIGS. 5 and 6. At this stage, one of the cleaning membranes 570 is moved forward by the driving element 573 into a position in front of the needles 12 of the chamber 530 (as illustrated in FIGS. 5 and 6) At resting position, the needles 12 do not reach or touch the membrane 570 enabling the cleaning pins 520 to be inserted into the lumen of each of the needles 12 by pushing and further unlocking the cleaning plunger 510, thus, preventing the penetration of any tissue parts and/or debris into the lumen of the needles 12 after and/or during the resection procedure. At this step irrigation liquid flows through the inlets (not shown) of the resectoscope in the direction 610 washing the needles 12 blocked by the cleaning wires 520 as is shown in FIG. 6.

When the delivery catheter 590 is pushed distally within the endoscopic sheath tube 591 the needles 12 penetrate the membrane 570. Each needle of the needles 12 actually perforates or punches a hole in the membrane 570 as it penetrates the bladder wall 350. In turn, the cleaning pins 520 penetrate into the lumen of the needles 12, as explained hereinabove without touching the surface of the region of tissue to be treated. As the delivery catheter 590 is even further pushed distally within the endoscopic sheath tube 591 the top part of the needle array 10 pushes the membrane 570 against the surface of the tissue region being treated. The maximal depth of penetration of the needles 12 into the treated tissue is limited by the two stopper pins 598 attached to the sheath tube 591. By pushing the injection rod 582 to a known distance, which is determined by the volume of the therapeutic agent solution 370 to be injected, the position of the injection piston 584 changes thus enabling injection of the solution 370 into the target tissue through the needles 12.

After the solution 370 is delivered to the target tissue of the bladder wall 350, the delivery catheter 590 is moved backwards within the endoscopic sheath tube 591 and retracted in a direction away from the tissue of the bladder wall 350 as is shown in FIG. 6. During the retraction movement of the delivery catheter 590 within the endoscopic sheath tube 591, the outer surfaces of the needles 12 are cleaned of tissue debris and/or adhering cancerous cells (if present) as the needles 12 pass through the membranes 570. The tight pass through the cleaning membrane 570 scrapes and removes adhering material and/or cells. After the cleaning action is accomplished and the needles are fully retracted within the endoscopic sheath tube 591, the used (perforated) membrane 570 is moved away (sideways) from its frontal position and replaced by a new intact membrane 570 by suitably activating the driving element 573. In the next step, the injection piston 584 is proximally retracted within the endoscopic sheath tube 591.

The cleaning pins 520 are then inserted into the lumen of each of the needles 12 such that at least a part of the cleaning pin 520 equal or longer than the length of the needle 12 is inserted into and passes through the lumen of the needles 12 and protrudes to a certain extent beyond the tip of the needle 12. Such cleaning of the inner surfaces of the lumen of the needles 12 by means of the cleaning pins 520 allows the expelling of all tissue debris and/or cellular material out of the needle's lumen. The parts of the cleaning pins 520 protruding out of the needles 12 as well as the outer part of the needles 12 are additionally cleaned and washed by the flow of the irrigation liquid (e.g. saline), transferred through the standard resectoscope inlets (not shown) in the direction schematically represented by the arrow 610 in FIG. 6. In accordance with an additional embodiment of the present invention, ultrasonic energy might also be used for the cleaning procedure of the inner bores of the needles 12, for example, by inserting a slim ultrasound horn (not shown) into the delivery catheter 590 instead of the cleaning plunger 510 it will transfer ultrasonic energy towards the needles 12. Ultrasonic cleaning has proven to be the most effortless, quick and efficient method known today.

Reference is now made to FIG. 7, which is a schematic diagram illustrating a region of tissue to be treated and the relative footprint of the area treatable by one treatment cycle of the multi-needle catheter device system 300 of FIG. 4. The area 710 schematically represents the region of tissue or organ that needs to be treated. The plurality of circles 720 schematically represent areas already treated by the tip of the catheter, and the cross-hatching circles 730 schematically represent areas that have not yet been treated by the multi-needle catheter device system 300. During the procedure, dyed, pigmented or colored agents or solutions are advantageous by enabling easy visual detection of the treated areas(colored) in comparison to untreated areas (not colored). The circles 730 thus schematically represent possible potential locations where the multi-needle catheter device system 300 may be placed on the surface of the target tissue in order to provide sufficient coverage of the target area by the drug or substance.

Reference is now made to FIG. 8A and 8B which are schematic part cross-sectional diagrams illustrating two operational states (before the injection procedure and during the injection procedure, respectively) of part of a protected multi-needle array catheter system, in accordance with an embodiment of the present invention. As described below, the protected multi-needle catheter system 800 may have a needle protection system in accordance with the present invention without need for a sheath tube.

In FIG. 8A, part of a protected multi-needle catheter system 800 is illustrated. The catheter system 800 includes an acceleration chamber 810 having a needle holder disc 11 sealingly attached thereto. The internal space 812 of the acceleration chamber 810 may be filled with the solution 370 of a drug and/or a substance and/or a therapeutic agent, and/or a diagnostic agent, as disclosed in detail hereinabove. The needle holder disc 11 is attached at the distal end of the chamber 810 and includes hollow needles 12 suitably attached thereto, such that the solution 370 may be injected through the needles 12 when pressure is applied to the solution 370.

The needle protecting system of the protected catheter system 800 includes a protector 830 and springs 835. The protector 830 covers the needles 12, during insertion into the resectoscope and protects the internal area of the bladder while re-locating the catheter during multiple procedures. The protector 830 may be advantageous in the case of bladder cancer treatment where multiple injections, causing bladder tissue penetrations, with the needles 12 of the catheter system 800 at multiple tissue regions are required in order to ensure proper agent or drug dispersal in the area or region to be treated (see FIG. 7 for the schematic representation of multiple injected locations covering a larger area in need to be treated). In the case of such multiple injection sites within a larger bladder treatment region, such multiple tissue penetrations and injections may result in occasional removal of the tissue debris, which may block the needles 12 or stick in the area between the needles 12. The protector 830 may prevent occasional scratching or injury of the tissue while re-locating the catheter system to the next site to be treated.

The protector 830 includes orifices 837 which are located and suitably aligned over each of the needles 12. The number of the orifices 837 is preferably equal to the number of the needles 12. When the protector 830 is in rest position, each of the needles 12 is located within the corresponding orifices in such a manner that the protector 830 completely covers all needle tips.

The needle protecting system of the protected catheter system 800 also includes the springs 835, which connect the protector 830 to the top part of the needle holder disk 11.

The catheter system 800 includes a plunger 820 with the piston 850 at its distal part. The plunger 820 has o-rings 860, which fit into the circumference of the piston 850 and provides a leak tight seal between the piston 850 and the walls of the delivery catheter 840.

At resting position, the catheter system 800 is positioned adjacent to or in contact with the region to be treated on the surface of the bladder wall 350. By moving the catheter system 800 distally, the protector 830 first reaches the treatment area. Further pushing of the catheter system 800 is defined by the interaction between both resistances (elasticity) (treatment area and springs 835). As soon as sufficient force will be applied to push the catheter system 800, the protector 830 will be retracted, pressing the springs 835 and uncovering the needles 12. The elasticity of the springs 835 may be chosen in such a manner that it may be pressed in interaction with a bladder tissue having average physical properties,

In turn, the needles 12 penetrate the bladder wall 350 to a defined depth determined by the overall length of the sidewall protector 830, springs 835 and the length of the needles 12 itself (see FIG. 8B) Additional stopper is located at the distal end of the catheter (not shown).

By manually or automatically pushing the plunger 820 to a predetermined distance forward (in a direction generally towards the needles 12) a certain volume of therapeutic or diagnostic agent solution 370 is forced through the needles 12 and injected into the target tissue of the bladder wall 350.

After the delivery of the therapeutic or diagnostic solution 370 to the target tissue 350, the catheter system is still in working position. Returning the catheter to its resting position requires the removal of the catheter system 800 from the treated area. At this moment, the springs 835 push the protector 830 forward, thus removing debris of the treated bladder tissue, which may block the needles 12 and/or be stuck in the areas between the needles 12; the needles 12 are again covered and protected.

Before the catheter system can be reused to deliver another aliquot of therapeutic or diagnostic agent 370 to another part of the bladder wall 350, the catheter system 800 is positioned adjacent to or in contact with the new region to be treated on the surface of the bladder wall 350. The required volume of the therapeutic or diagnostic agent solution 370 is filled into the chamber 810 and in the distal part to the delivery catheter 840. Each injection delivers a required equal portion of the therapeutic or diagnostic solution 370 to the known effective surface of the target tissue 350. The required injected volume for each injection is defined by the type of the drug or other agent dissolved or suspended in the solution 370 and may be controlled by the dispensing system, located at the proximal end of the needle catheter system 900 (not shown here) or by a standard syringe. The effective area of each injection is defined by the effective surface of the needle array 10 as it was explained above in description of FIG. 3A and FIG. 3B.

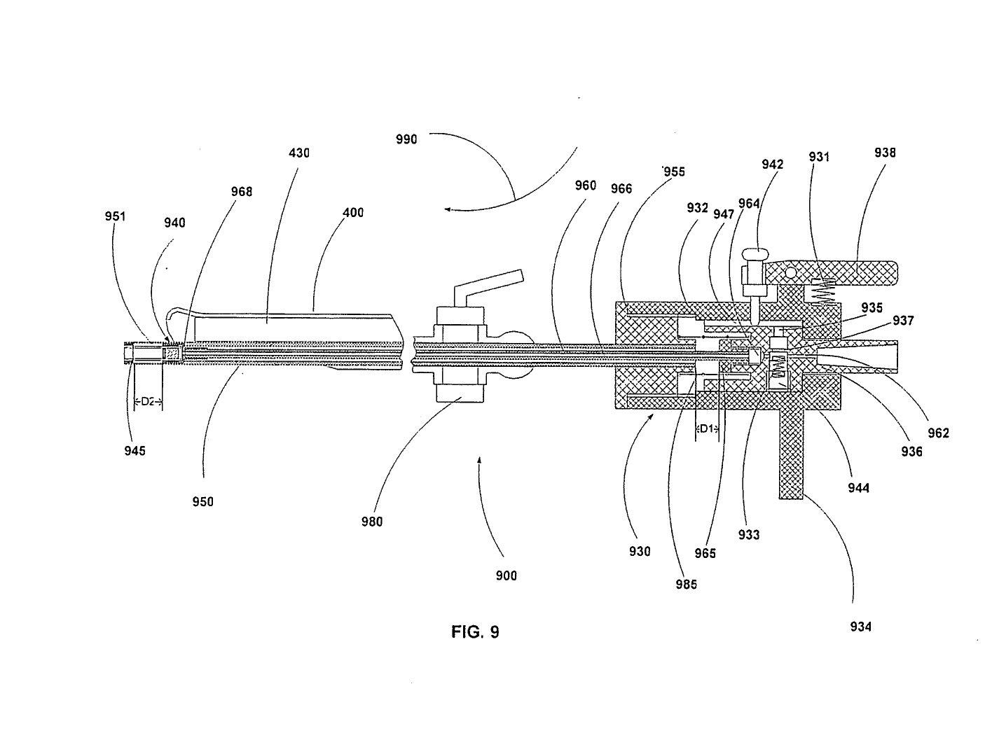

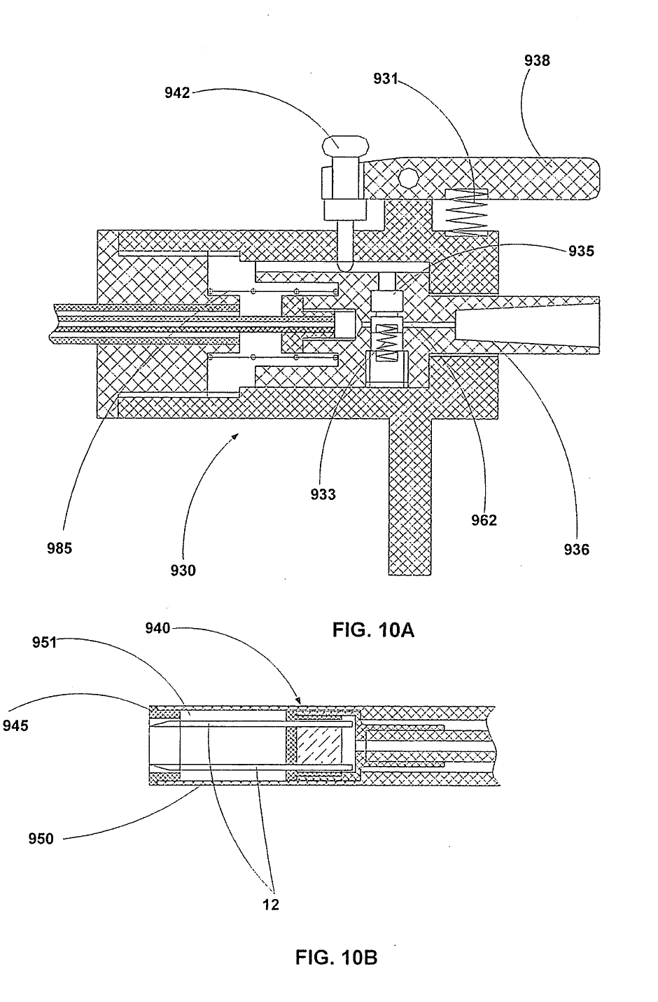

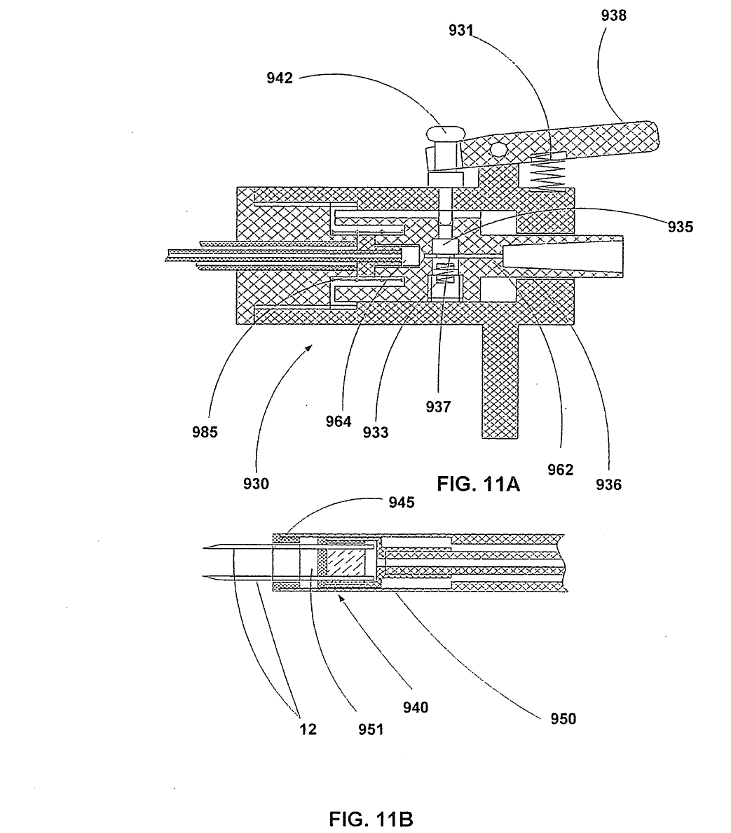

Reference is now made to FIG. 9 which is a schematic part cross-sectional diagram illustrating a manually or automatically driven multi-needle catheter device 900 useable in conjunction with an endoscope in accordance with an another embodiment of the present invention; more specifically an endoscope 400, for the injection of therapeutic or diagnostic agents to the bladder wall if it is suspected, for instance, of containing cancerous cells. The catheter device 900 includes a luer end handle 930 at its proximal end, a multi-needle head 940 and a protector 945 at its distal end; a sheath tube 950 and a delivery tube 960 are the tubing part of the catheter device 900. A multi-needle head 940 (see FIGS. 10B and 11B) is suitably attached or connected to the distal part of the delivery tube 960. The protector 945 is suitably attached or connected to the distal part of the sheath tube 950. The delivery tube 960 ends, in its proximal side, with bushing A 965 suitably attached or connected to it and the sheath tube 950 ends with bushing B 955 suitably attached or connected to it. In more details all elements of the catheter device 900 will be described below in further embodiments (FIGS. 10 and 11). The luer end handle 930 consists of an enclosure 932, a holder 934, a luer end piston 936, a button 938 and a fixation pin 942. The luer end handle 930 also includes two springs, a button spring 931 and a valve spring 933; as well as a valve 935 with groove 937, a cap 944 and slit 947.

The multi-needle catheter device 900 may be fully or partly disposable. In case of a fully disposable device the multi-needle catheter device 900 is composed of one single unit, while a partially disposable catheter device 900 is composed of at least two parts. The first part of the partially disposable multi-needle catheter device 900 is a disposable part which includes the following elements: a multi-needle head 940, a delivery tube 960, a sheath tube 950 and two bushings: bushing A 965 and bushing B 955. Suitably connected, the above mentioned parts assemble the disposable unit. The second part of the partially disposable multi-needle catheter device 900 is the non-disposable luer-end handle 930. In case of treating another patient or the same patient with another drug solution, the disposable part is replaced, while the non-disposable part is sterilized.

The therapeutic solution is inserted via the Luer- end piston 936, delivering input channel 962, intermediate chamber 964 and the inner lumen 966 of the delivery tube 960 to finally fill the hollow space of the chamber 968 of the multi-needle head 940. The delivery tube 960 is inserted into the sheath tube 950 which ends by a protector 945, limiting the penetration depth of the needles located on top of the multi-needle head 940 into the tissue. The endoscope 400 provides the means for observing, and guiding the catheter during the procedure via a monitor (not described) connected to an optical inlet (not shown) of the telescope 430.

The catheter device 900 is installed from the proximal part of the endoscope, via a mechanical bridge 980 (not shown in detail), to its distal part. The arrow 990 shows an installation direction.

Further to this embodiment, the application of the therapeutic agent installation for the purpose of bladder cancer treatment is performed in conjunction, and immediately subsequent to TUR (the removal of superficial bladder cancer tumor) from the bladder wall In order to cover a relatively large surface of the bladder, the treatment provider should be able to identify the tissue area already treated so as to provide optimal treatment coverage. The multi-needle catheter device 900 may be rigid or flexible and may preferably be fully disposable. The proximal part of the catheter may be connected via a suitable connector to a standard medical syringe or to any other type of manual or automatic dispensing system, as is known in the art.

At resting position, the distal end the multi-needle catheter device 900 is positioned adjacent to or in contact with the region to be treated, on the surface of the bladder wall, by directing the luer end handle 930 distally by means of relocating the holder 934. Once the treatment area is touched, the next step is pricking the bladder wall, using the catheter device, to a predetermined depth. The procedure of “touching” the treatment area may be made in a conventional way: the treatment provider personal watches the monitor and locates the target area and then manually approaches it and feels the moment of touch. Optionally, this “touching” procedure may be made automatically with touching sensor located at the proximal tip of the catheter 900. Such a “touching” sensor may be based on, for instance, impedance sensor yet may be any other kind of sensors known in the art.

For the purpose of pricking, the luer-end piston 936 is pushed distally, until it is stopped at the edge of bushing B 955. This push is performed by means of standard medical syringe or any other type of manual or automatic dispensing system, as is known in the art, which is used with the catheter 900. At this moment, the retracting spring 985 is pressed, the delivery tube 960, with the multi-needle head 940 attached to its distal part, moves freely forward within the sheath tube 950, the multi-needle head 940 approaches from the protector 945 and pricks the area to be treated.

In turn, the fixation pin 942, forced by the button spring 931, pushes down the valve 935 and locks the luer-end piston 936 in this position. The distance D1 between the edges of bushing B 955 and the edges of bushing A 965 exactly equals the required injection depth by means of the multi-needle head 940 (see FIGS. 10B and 11B for reference). In order to prevent the impact between the protector 945 and the basis of the multi-needle head 940, the distance D2 between the protector 945 and the basis of the multi-needle head 940 is larger than D1 for a certain value ΔD whereas D2=D1+ΔD.

During operation, at this specific step, the treatment provider personal receives a signal that confirms passing the distance D1 by means of, for instance, audible signal (like “clicking” a pen's cap). Such a signal may be also any other kind of an alarming signal—sound, optical etc. The measurement of the distance D1 (or D2 or both) may be also made by means of different sensors, like electronic impedance, capacitance etc. Further, in turn the delivering input channel 962 is connected to the intermediate chamber 964 and to the hollow lumen 966 of the delivery tube 960 by means of the groove 937 of the valve 935, which enables the delivery of the therapeutic agent in working position and disables the delivery in the case of resting position. Only after receiving the confirmation signal the treatment provider personal starts the next step which is the delivery of therapeutic or diagnostic solution to the target tissue. Optionally, the required injection depth may be varied by changing the distance D1 at the proximal part of the multi-needle catheter device 900 and adapting the needle length to the maximal requirement for a known injection depth. Taking into account the distance differences between the protector 945 and the basis of the multi-needle head 940, the needle length will be the sum of both. Changing the injection depths may be controlled by any suitable type of sensor, like mechanical, optical, electrical etc.

After the therapeutic or diagnostic solution is delivered to the target tissue, the catheter system is still in working position. Returning the multi-needle head 940 to its resting position requires pushing the button 938, which raises the fixation pin 942. At this moment, the valve 935, forced by the valve spring 933, returns to its resting position and blocks the delivering input channel 962, preventing in such a way the undesired dripping of the therapeutic agent. In turn, luer-end piston 936 is retracted in a direction away from the tissue by means of the retracting spring 985 to its resting position, simultaneously retracting the multi-needle head 940 back proximally via the protector 945 to its resting position within to the hollow lumen 951 of the sheath tube 950. During the retraction movement of the multi-needle head 940 within the sheath tube 950 the multi-needle head 940 tightly passes the protector 945, which cleans the effective working area out of tissue debris and/or adhering cancerous cells (if present),

Retracting the catheter 900 to its resting position requires the removal of the catheter 900 from the treatment area. Before the catheter system can be reused to deliver another aliquot of therapeutic or diagnostic agent to another part of the bladder wall, the catheter system 900 is positioned adjacent to or in contact with the new region to be treated on the surface of the bladder wall. Each injection delivers a required portion of the therapeutic or diagnostic solution to the known effective surface of the target tissue. The effective area for each injection is defined by the effective surface of the needle array 900. For instance, if a 27 F (9 millimeters) resectoscope or cystoscope is used for the procedure; a space of only 15 F (4.5 millimeters) diameter may be used for inserting the needle catheter of the invention. Taking into account the ellipsoidal shape of the endoscope, this allows the insertion of a catheter having a circular cross-section with a diameter of approximately 3 millimeters.

The required injected volume for each injection is defined by the type of the drug or other agent dissolved or suspended in the solution and may be controlled by a dispensing system, located at the proximal end of the needle catheter or by a standard syringe.

Reference is now made to FIGS. 10A and 10B which are schematic cross-sectional diagrams illustrating two different parts of a multi-needle catheter system, in accordance with an embodiment of the present invention before the injection procedure.

In FIG. 10A, the luer end handle 930, part of a multi-needle catheter device 900, is illustrated at resting position. At resting position, before the injection takes place, the luer-end piston 936 is located at its backward position, the button 938 presses the button spring 931, the fixation pin 942 is at its upper position and the retracting spring 985 is at its resting non-pressed position. At this moment, the valve 935 is forced by the valve spring 933, which is also located at its upper position, blocking the delivering input channel 962 and in such a way preventing undesired dripping of the therapeutic or diagnostic agent.

In FIG. 10B, the distal part of the multi-needle catheter device 900 is illustrated at resting position. At resting position, before injection, the multi-needle head 940 and the needles 12 are located within the hollow lumen 951 of the sheath tube 950, fully covered by the protector 945 and by the sheath tube 950.

Reference is now made to FIGS. 11A and 11B which are schematic cross-sectional diagrams illustrating two different parts of the multi-needle catheter system, in accordance with an embodiment of the present invention during the injection procedure.

In FIG. 11A, the luer-end handle 930, part of a multi-needle catheter device 900, is illustrated during the injection stage. At working position, during injection, the luer-end piston 936 is pushed distally towards to the tissue, fixation pin 942 is in its lower position, the button 938 is in its upper position, button spring 931 is at its rest status and the retracting spring 985 is at pressed status. At this moment, the valve 935 is forced by the fixation pin 942 and been pushed down pressing the valve spring 933 and causing it to make the “click” audible signal, which signals to the treatment provider personal a confirmation of completing the injecting procedure into the required depth. In turn, groove 937 connects the delivering input channel 962 and the intermediate chamber 964, allowing a smooth therapeutic or diagnostic agent flow,

In FIG. 11B, the distal part of a multi-needle catheter device 900 is illustrated during the injection stage. At working position, during injection, the multi-needle head 940 with the needles 12 is pushed distally within to the hollow lumen 951 of the sheath tube 950, passing the protector 945, to finally fully exposing the needles 12 and forcing it to penetrate the tissue.

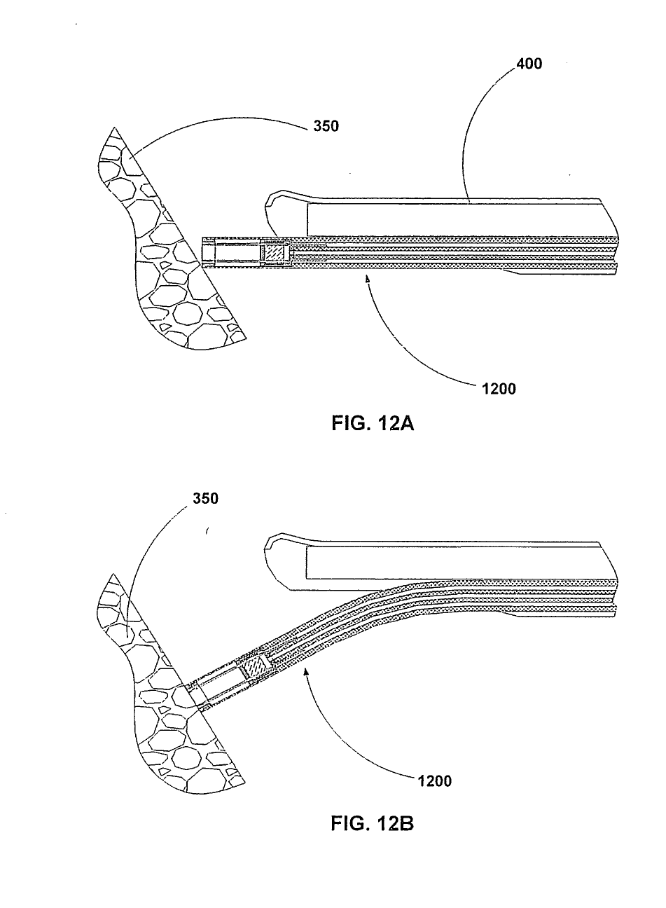

Reference is now made to FIGS. 12A and 12B which are schematic cross-sectional diagrams illustrating two different stages in the operation of the semi-flexible multi-needle catheter system in accordance with an embodiment of the present invention.

In FIG. 12A, the semi-flexible multi-needle catheter system 1200 is inserted into the endoscope 400. The semi-flexible multi-needle catheter system 1200 is used for obtaining the right angle or beveled for pricking the tissue 350, which is very important for the treatments made in specific areas of the bladder. The specific areas are located on both sides, top and bottom parts in relation to the central axis and hardly reachable by means of a rigid catheter. Such semi-flexible or flexible multi-needle catheter 1200 may be manufactured out of plastic tubing with Hardness—Durometer about D 50 (e.g. PTFE or FEP tubing). In FIG. 12A the semi-flexible multi-needle catheter 1200 is positioned adjacent to or in contact with the region to be treated on the surface of the bladder wall 350 before injection.

In FIG. 12B the semi-flexible multi-needle catheter system 1200 is illustrated during the injection stage. Due to the flexibility of the semi-flexible multi-needle catheter system 1200 during injection to the bladder wall 350 the catheter is bended allowing more accurate injection ability.

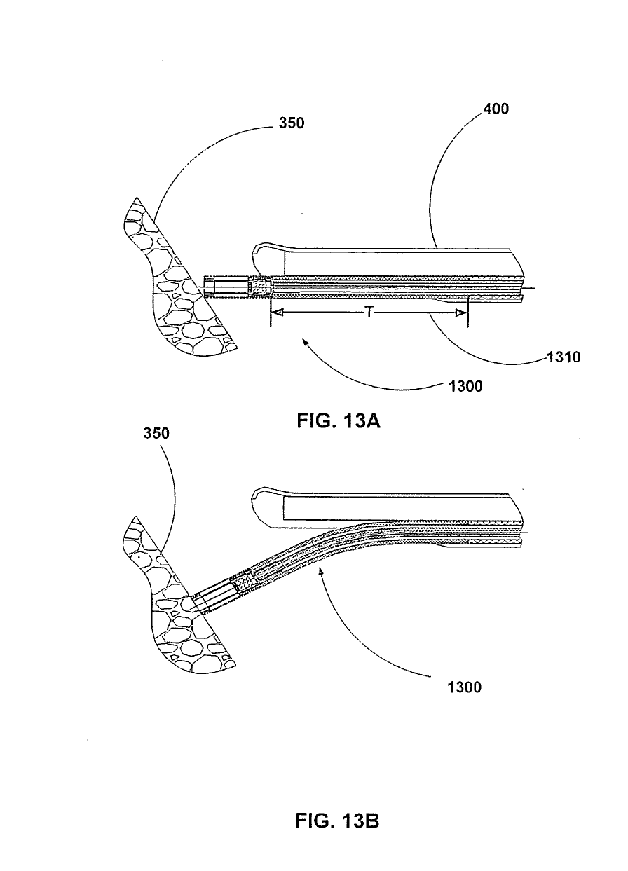

Reference is now made to FIGS. 13A and 13B which are schematic cross-sectional diagrams illustrating two different stages in the operation of the partially flexible multi-needle catheter system in accordance with an embodiment of the present invention.

In FIG. 13A, the partially flexible multi-needle catheter system 1300 is inserted into the endoscope 400. The partially flexible multi-needle catheter system 1300 is used for obtaining the right angle or beveled for pricking the tissue 350 as it was explained in the previous embodiment. The difference is that the partially flexible multi-needle catheter system 1300 has at least one flexible part 1310 as it is shown in FIG. 13A and noted by “T”. The hardness parameters of the flexible component or components, and its lengths may be optionally varied during the design for obtaining the optimal bending angle while pricking a tissue. In FIG. 13A the partially flexible multi-needle catheter system 1300 is positioned adjacent to or in contact with the region to be treated on the surface of the bladder wall 1310 before injection.

In FIG. 13B the partially flexible multi-needle catheter system 1300 is illustrated during the injection stage, Due to the flexibility of the flexible part—the tip of the partially flexible multi-needle catheter system 1300 is bended during the injection procedure to the bladder wall 350 thus allowing a better positioning as well as a more precise and accurate injection.

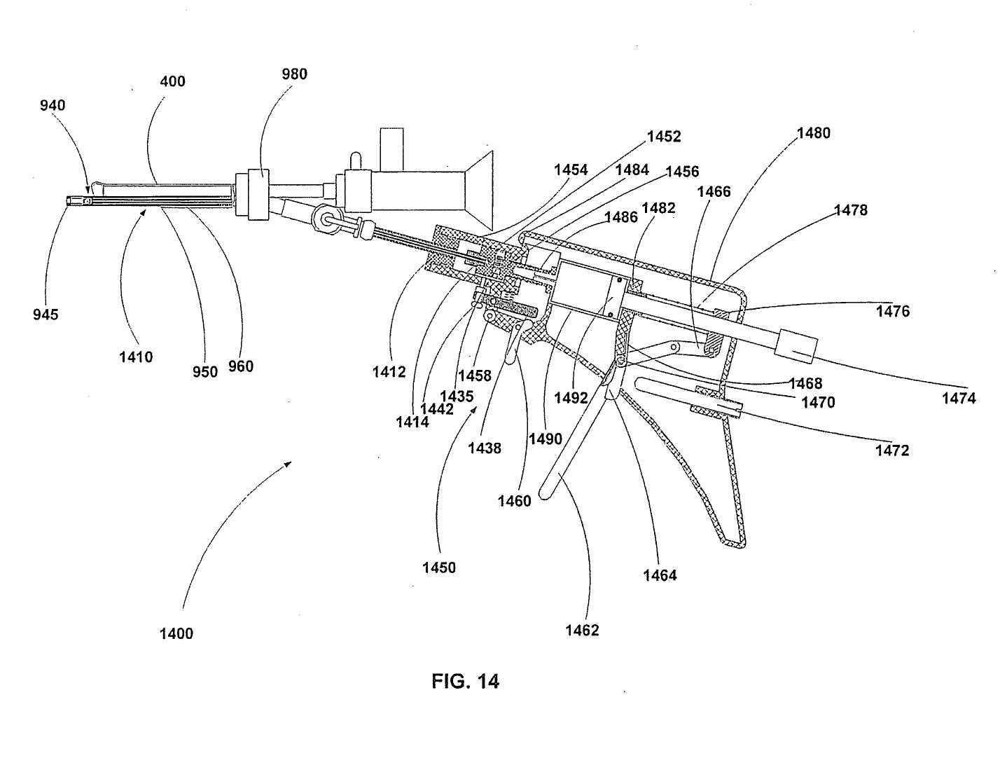

Reference is now made to FIG. 14 which is a schematic cross-sectional diagram illustrating a manually or automatically driven injector 1400 which includes an injector handle 1450 and a multi-needle catheter 1410 useable in conjunction with an endoscope, more specifically a resectoscope or cystoscope 400, for the injection of therapeutic or diagnostic agents to the bladder wall if it is suspected, for instance, of containing cancerous cells. The injector 1400 consists of two main parts: a re-usable injector handle 1450 and a disposable multi-needle catheter 1410.

The multi-needle catheter 1410 is the disposable part of the injector 1400 that includes a multi-needle head 940 and a protector 945 at its distal end; a sheath tube 950 and a delivery tube 960 are the tubing part of the catheter 1410. A multi-needle head 940 is suitably attached or connected to the distal part of the delivery tube 960. The protector 945 is suitably attached or connected to the distal part of the sheath tube 950. The delivery tube 960 ends, in its proximal side, with a bushing 1414 suitably attached or connected to it and the sheath tube 950 ends with a flange 1412 suitably attached or connected to it.

The injector handle 1450 consists of a catheter enclosure 1452 with a cap 1454, a luer-end piston 1456, a button 1438, a fixation pin 1442 and a valve 1435. The catheter enclosure 1452 is connected to the injector handle enclosure 1480 by means of axis 1458 and click 1484. Levers 1462, 1464 and 1466 compose a lever system with rotation axis 1470, the lever system is located within the injector handle enclosure 1480. The lever 1462 is attached to a bracket 1482 by means of torsion spring 1468. A regulator 1472, a rod 1474 with a driver 1476 and a return spring 1478 are related to the dispensing part of the injector handle 1450 as well as a disposable capsule 1490 with piston 1492. The trigger 1460 controls the button 1438.

At resting position, the rod 1474 is in its starting position within the injector handle 1450 and the regulator 1472 fixates the required dosage per injection. At this moment, the cap 1454, which is located within the catheter enclosure 1452, is opened, the bushing 1414 is placed into the luer-end piston 1456 and the cap 1454 is closed and fixes the flange 1412 within the enclosure 1452. In turn, click 1484 is pushed backward, which allows the rotation of the injector handle enclosure 1480. At this moment, the access to the proximal end of the luer-end piston 1456 is opened, which allows the placement of the disposable capsule 1490 within the injector handle 1450 and the connection of the capsule 1490 to the luer-end piston 1456. Finally, the injector handle enclosure 1480 is closed, fixed by means of a click 1484, and the injector 1400 is ready for injection.

Once the injector 1400 is in its working status, the multi-needle catheter 1410 is inserted into the endoscope 400 via a mechanical bridge 980 and is positioned adjacent to or in contact with the region to be treated on the surface of the bladder wall, by directing the injector handle enclosure 1480 distally. Once the treatment area is in touch, the next step is pricking the bladder wall, using the catheter device, to a predetermined depth. The procedure of “touching” the treatment area may be made in a conventional way, the treatment provider personal watches the monitor and locates the target area and then manually approaches it and feels the moment of touch. Optionally, this “touching” procedure may be made automatically with touching sensor located at the proximal tip of the catheter 1410. Such a “touching” sensor may be based on, for instance, impedance sensor yet may be any other kind of sensors known in the art.

For the purpose of pricking, the lever 1462 is pressed, which in turn pushes the disposable capsule 1490, the luer-end piston 1456 and the multi-needle catheter 1410 distally until the bushing 1414 is stopped by the edge of the flange 1412. At this moment, the retracting spring 1486 is pressed; the multi-needle head 940 approaches from the protector 945 and pricks the area to be treated.

In turn, the fixation pin 1442 pushes down the valve 1435 and locks the luer-end piston 1456 at this position. The distance between the edges of bushing 1414 and the flange 1412 equals exactly the required injection depth by means of the multi-needle head 940 as it was explained above in FIG. 9. After receiving the confirmation signal in regard to completing the pricking step, as explained above for FIG. 9, the treatment provider personal starts the next step which is the delivery of therapeutic or diagnostic solution to the target tissue.

Further, the delivery stage of the injection starts by “breaking” the lever 1462 relatively to the bracket 1482; by keeping the movement of the lever 1462, the lever 1464 is pressed and rotated along the axis 1470 At this moment the lever 1466 and driver 1476 move distally, the driver 1476 locks the rod 1474 and pushes it distally to a distance, which is set by means of the regulator 1472. In turn, the spring 1478 is being pressed. Finally, the rod 1474 pushes the piston 1492 of the disposable capsule 1490 for the delivery of the required dosage of the diagnostic or therapeutic solution.

After the therapeutic or diagnostic solution is delivered to the target tissue, the injector 1400 is still in working position. Returning the lever system and the driver 1476 to its resting position requires the release of the lever 1462 and the spring 1478. At this moment, the rod 1474 and the piston 1492 are kept in its position for the next delivery of therapeutic or diagnostic agent. Returning the multi-needle head 940 to its resting position requires pushing the button 938 by means of the trigger 1460, which releases the fixation pin 1442. At this moment, the valve 1435 returns to its resting position and blocks the delivering input, preventing in such a way the undesired dripping of the therapeutic or diagnostic agent. In turn, the luer-end piston 1456 is retracted in a direction away from the tissue, by means of the retracting spring 1486, to its resting position, simultaneously retracting the multi-needle head 940 back proximally via the protector 945 to its resting position in a way as it was explained in FIG. 9. During the retraction movement of the multi-needle head 940, the multi-needle head is cleaned as explained above. Retracting the catheter 1410 to its resting position requires the removal of the catheter 1410 from the treatment area. Before the injector 1400 can be reused, to deliver another dose of therapeutic or diagnostic agent to another part of the bladder wall, the injector 1400 is positioned adjacent to or in contact with the new region to be treated on the surface of the bladder wall.

The required injected volume for each injection is defined by the type of the drug or other agent dissolved or suspended in the solution and is controlled by the dispensing system, located within the re-usable injector handle 1450. In order to deliver the next portion of the drug solution to another part of the bladder wall, the delivery stage of the injection is repeated. The piston 1492 and the rod 1474 are kept in its position following the last release of the diagnostic or therapeutic agent. In a case of multi-injection procedure, the capsule 1490 may be replaced with additional capsule. For these purposes, the rod 1474 is retracted backwards to its resting position, the click 1484 is released, and the injector handle enclosure 1480 is opened.

The refilling procedure may be made in different possible ways. For instance, by using internal reusable chamber (instead of the disposable capsules) with a gateway for a standard syringe or, in contrary, by placing the standard syringe into the injector handle enclosure 1480.

In order to use the injector 1400 for additional treatment (another patient or for the same patient but different drug), the disposable multi-needle catheter 1410 is replaced by new one, and the reusable injector handle 1450 is sterilized.

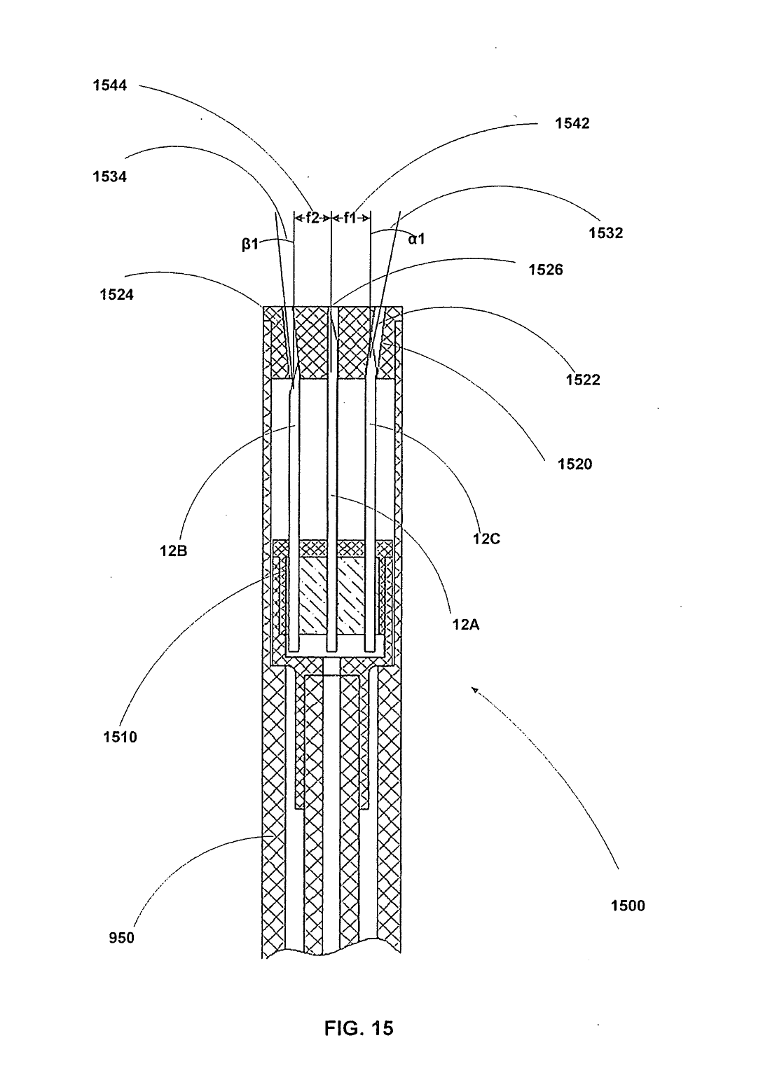

Reference is now made to FIG. 15 which is schematic cross-sectional diagrams illustrating the distal part of the multi-needle catheter system with an angled channel protector, in accordance with another embodiment of the present invention.

In FIG. 15, the distal part of a multi-needle catheter system 1500 is illustrated before the injection stage. A multi-needle head 1510 is built out of number of needles, a central needle 12A, and side needles 12B and 12C, which have different lengths. A protector 1520, located at the end of sheath tube 950 has at least two channels within its body, needed for aligning the needles during the injection procedure. At least two of the channels have different angles in respect to the central axis, as is illustrated here angle al referred by 1532 for channel 1522 and angle β1, referred by 1534 for channel 1524. The central channel 1526 is straight. Because of such a construction, the distances between the needles point orifices are different: f1, referred by 1542 and f2, referred by 1544 Note here, that the angled channels force the needles to cover a larger volume, especially when the needles are in different lengths, or in another words, different penetration depths enable more homogeneous volumetric distribution of the drug solution within the treated area.

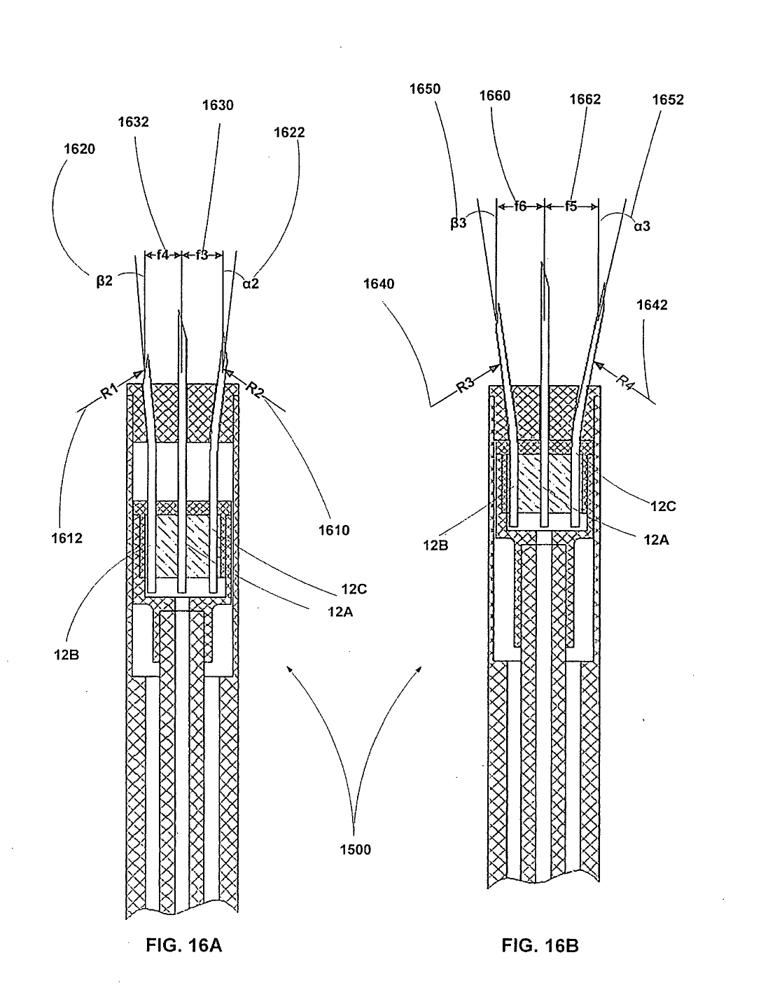

Reference is now made to FIGS. 16A and 16B which are schematic cross-sectional diagrams illustrating two different stages in the operation of the multi-needle catheter system with an angled channel protector in accordance with the present invention.

In FIG. 16A, the multi-needle catheter system with an angled channel protector 1500 is illustrated during the injection stage Needles 12A, 12B and 12C, are moved and exposed at a certain distance distally. At this moment the distance between the needles has been changed relatively to the starting position, and it is illustrated in FIG. 15. The distance f1 referenced in FIG. 15 as 1542 is less than the distance f3 referenced in FIG. 16A as 1630 and the distance f2 referenced in FIG. 15 as 1544 is less than the distance f4 referenced in FIG. 16A as 1632. An additional difference in the needle orientation appears as a deflection in angles α2, referenced in FIG. 16A as 1622 and β2 referenced in FIG. 16A as 1620 relative to each other.

In FIG. 16B the multi-needle catheter system with an angled channel protector 1500 is illustrated at the end of the injection stage, when needles 12A, 12B and 12C are maximally exposed distally. The relative orientation between the needle's orifices has changed relative to FIG. 16A distance f5 referenced in FIG. 16B as 1662 is greater than the distance f3 referenced in FIG. 16A as 1630 and the distance f6 referenced in FIG. 16B as 1660 is greater than the distance f4 referenced in FIG. 16A as 1632. In some embodiments, the relative deflection angles may also change: α3 referenced in FIG. 16B as 1652 may be greater than α2 referenced in FIG. 16A as 1622 and β3 referenced in FIG. 16B as 1650 may be greater than β2 referenced in FIG. 16A as 1620. In addition, in some embodiments, the radius of curvature of the needle may change during the injection process as is shown in FIGS. 16A and 16B: radius R1 referenced in FIG. 16A as 1612 may be greater than the radius R3 referenced in FIG. 16B as 1640 and radius R2 referenced in FIG. 16A as 1610 is greater than radius R4 referenced in FIG. 16B as 1642.

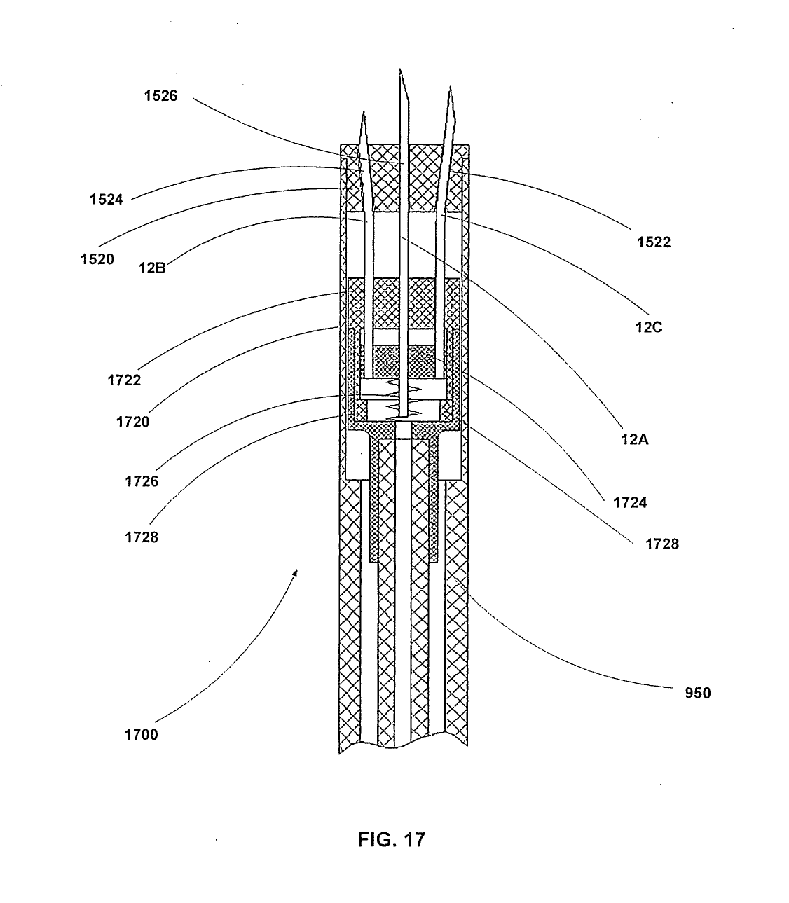

Reference is now made to FIG. 17 which is schematic cross-sectional diagram illustrating the distal part of the dynamic multi-needle catheter system with an angled-channel protector, in accordance with an embodiment of the present invention.

In FIG. 17, the distal part of the dynamic multi-needle catheter system 1700 is illustrated during the injection stage, A multi-needle head 1720 may be composed of two main parts: the dynamic disk 1724 and the static disk 1722. The static disk 1722 includes at least one needle, as it illustrated in FIG. 17—needle 12A and the dynamic disk comprises of at least one needle—needles 12C and 12B. The dynamic disk 1724 may change its position within the multi-needle head 1720 during the injection, yet, its most proximal position is limited by a pair of stoppers 1728. The dynamic disk 1724 is connected to the lower part of the multi-needle head 1720 by a flexible element 1726, which is illustrated in FIG. 17 as a retractable spring, but it may be any kind of flexible membrane, or different types of springs.

Similarly to the multi-needle catheter system with an angled channel protector illustrated in FIGS. 16A and 16B, the dynamic multi-needle catheter system 1700 is ended, at its distal part, by a protector 1520 which is located at the distal end of the sheath tube 950 with the angled channels 1522, 1524 and 1526. The difference between the two types of the catheter systems is by its injection action: the needles of the multi-needle head of static catheter 1500 (see FIGS. 15, 16A and 16B) changes its relative orientation along the transversal axis, while the dynamic catheter 1700 (see FIG. 17) changes its relative orientation along both, transversal and longitudinal, axis. This feature allows the adaptation of the integral needle array shape to the mechanical properties of the treated tissue. The more resistive the tissue is, the sharper the integral needle array shape becomes. It is regulated by the relative displacement of the two disks (the dynamic disk 1724 and the static disk 1722).

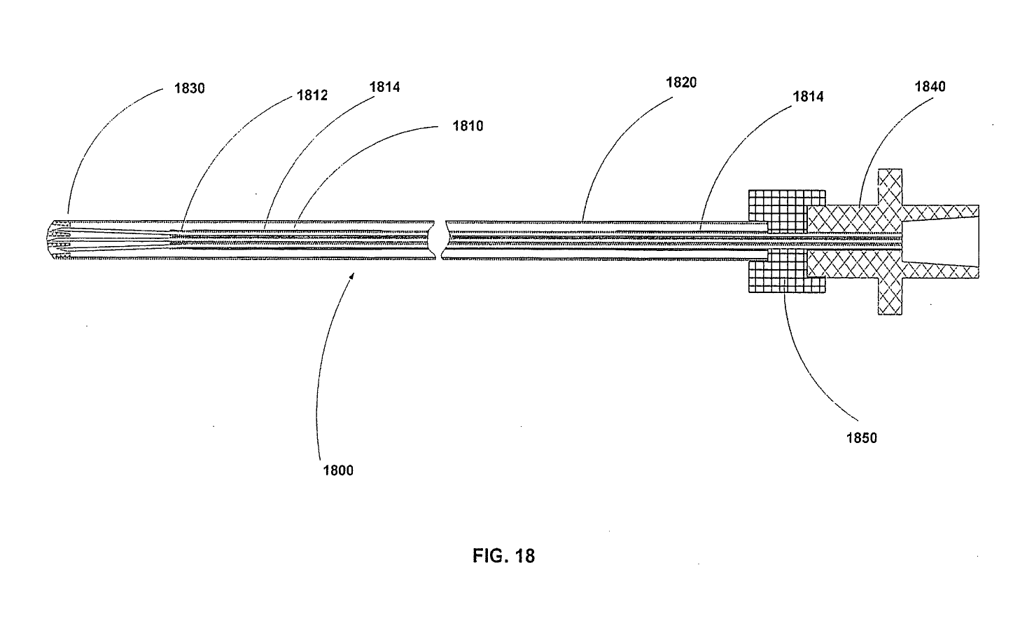

Reference is now made to FIG. 18 which is schematic cross-sectional diagram illustrating a multi-catheter delivery system, in accordance with yet another embodiment of the present invention.

In FIG. 18 the multi-catheter delivery system 1800 consists of a sheath tube 1820, which is ended, at its distal part, by a protector 1830 and at its proximal part is fastened to the bushing 1850, Luer adapter 1840 and a multi-catheter array 1810. The multi-catheter array 1810 is composed of a number (at least two) of catheters, composed of a thin tube and a needle at its end. The catheters are firmly bundled together by means for example of a number (at least one) of heat-shrink tubes, as it is illustrated in FIG. 18 by 1814. Here, the heat-shrink tubes 1814 bundle the multi-catheter array 1810 at two locations. The multi-catheter array 1810 may be moved, within the sheath tube 1820. During injection, the multi-catheter array 1810 may be exposed through the protector 1830. Prior and post procedure, while inserting or withdrawing the device from the endoscope or between injections in a multi-injection mode the multi-catheter array 1810 may be located before the protector 1830 within the sheath tube 1820.

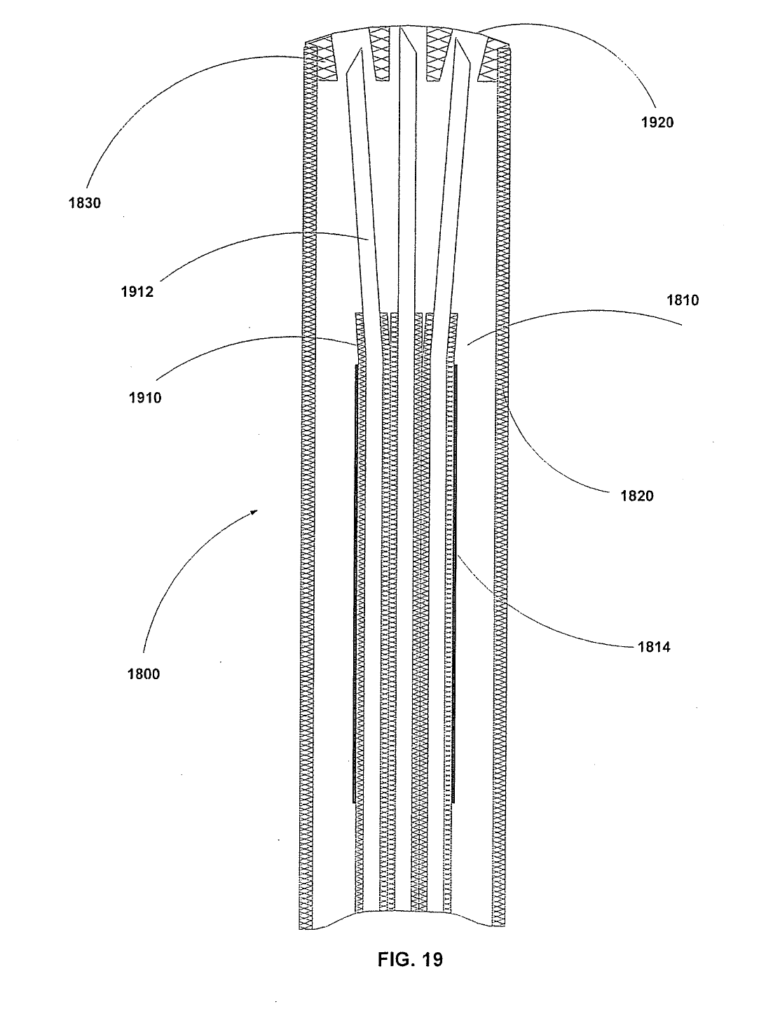

The multi-catheter array 1810 may be built, as was mentioned above, out of an array of catheters 1812 which is a thin tube with a needle at one end or out of an array of cannulas (shown on FIG. 19 in details). As it illustrated in FIG. 18, each catheter is separately fluidly connected to the drug dispensing system (either a syringe or an automatically or manually driven injector) via the luer adapter 1840. Such a multi-catheter delivery system 1800 is especially relevant for flexible endoscopes (for example Fiber-Urethro-Cystoscope by RICHARD WOLF GmbH), which has a relatively small working channel and 90°-180° bending angle at its distal part.

Reference is now made to FIG. 19 which is schematic cross-sectional diagram illustrating the distal part of the multi-catheter delivery system, in accordance with an embodiment of the present invention.

In FIG. 19 the distal part of the multi-catheter delivery system 1800, shown in FIG. 18, is illustrated here in more detail. Here, 1820 is the sheath tube, ended at its distal end by a protector 1830. The protector 1830 has at least one angled channel 1920 per needle, in similarity to the protector of the multi-needle catheter system 1500 shown in FIG. 15. The channels may have different declination angles or equal declination angles, in relation to the chosen application. As it was mentioned above, the multi-catheter array 1810 may consist of at least two thin catheters, each catheter is ended by a needle. FIG. 19 demonstrates a single catheter, 1910, with its needle 1912 which comprises the multi-catheter array 1810. As it was mentioned above, instead of at least a pair of catheters with needles, cannulas or any other hollow injection elements may be used, The members of the multi-catheter array 1810 may have the same length or may vary, in relation to the chosen application. The fastener element 1814, which bundles all members together, may be a heat-shrink tube or any other fastener, like a tube or tape.

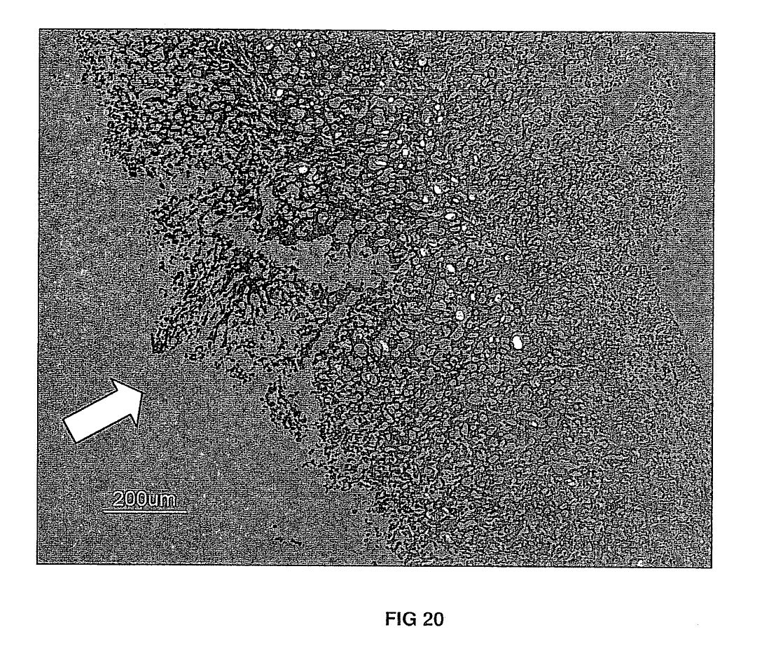

Reference is now made to FIG. 20 which a photomicrograph illustrating histology results taken from a C3H/eb mouse bladder tumor (MBT-2) after treatment with the multi-needle catheter of the present invention., MBT-2 is a well known murine cancer model suitable for studying superficial bladder cancer as described in. Nativ O., et al., entitled “Combined local bladder hyperthermia and intravesical chemotherapy for the treatment of high-grade superficial bladder cancer”, in Urology 2004; 63(3):466-471. A multi-needle catheter prototype was constructed, (similar to the catheter illustrated in FIG. 9) including 7 needles having a size of 30 G (310 microns outer diameter and 160 microns inner diameter).

The needles were uniformly distributed on a 2.0×2.5 millimeters area of a 7 needle holder. Two groups of needles were used. In the first group of 7 needles, each needle had a length of 1.1 millimeter (extending beyond the surface of the needle holder), and in the second group of 7 needles, each needle had a length of 1.5 millimeter (extending beyond the surface of the needle holder). All needles had a 15° beveled tip (Point Style 4 in accordance of the definition of Hamilton Company (www.hamiltoncomp.com)).

The therapeutic or diagnostic solutions used for urological treatments (or for treatment of other diseased organs or tissues) may be in the form of a solution of one or more agents or compounds or may be formulated as a suspension of solid particles of a drug suspended in a pharmaceutically acceptable vehicle or carrier. The capsules may contain a certain amount of the therapeutic or diagnostic agents for ensuring long term action (i.e. slow-release) as are well known in the art. All materials are delivered into the target tissue either in accordance with the manufacturer's instructions or after encapsulation.

Materials which are used for the degradable capsules production are mostly polymers and copolymers, like, for example, aliphatic polyesters based on lactic acid (PLA) or Polylactide-Glycolide copolymers (PLGA), as is disclosed in the following publications: N. Nill and J. Sandow “Two poly (D, L-Lactide-CO-Glycolide) 50:50 types: same polymer but different properties?”—(Intern. Symp. Control. Re. Biact. Mater. 23(1996); Dieter Bendix “Chemical synthesis of polyactide and its copolymers for medical applications,”—Polymer Degradation and Stability, 59 (1998)). The encapsulation of therapeutic agent may be also done by other technologies such as LBL (“layer-by layer”) of “Capsulution NanoScience AG”, (Berlin, Germany). For further applications, different pigments, dyes, coloring materials or florescence tags may be added to the capsules. The pigmentation enables visualization of the treated area by the surgeon during operation in order to ease the procedure.

The blank poly—DL-lactic acid (PLA) microcapsules were labeled with the fluorescent dye Nile Red and were used as a preliminary model to the microcapsules containing the therapeutic agents. The average capsule's size was in the 12-25 micron range.

The solution was prepared according to a known iv ratio of Gemzar® (Lilly France S.A., Fegersheim, France) (1:25) (dilution of 1 g of drug in 25 ml of 0.9% NaCl solution in water).The dosage was calculated in accordance with the effective treatment surface according to the drug's manufacturer's instructions. The saline including the capsules was injected into a tumor located on the mouse back after removing the skin. After injection the mice were sacrificed and the treated excised tumors were removed and fixed for further histology analysis. The histology analysis was performed by using frozen section procedure. For frozen sectioning, tissue fragments were snap-freeze in Isopentane which was pre-cooled in liquid nitrogen and immediately transferred and stored at −70° C.

The tumor fragments were adhered by O.C.T (frozen tissue matrix) to a metal stand of the cryostat (Leica CM1900) held at −20° C. Serial tissue sections of 5-20 micrometers each were cut in the cryostat for every 50 μm of tissue. Slides were examined using fluorescence microscope (Nikon ACT-1, Japan). The white points seen in FIG. 20 represent the Nile red fluorescent capsules injected into the tissue to a depth of approximately 1 millimeter. The tissue sample was first cut through the center of the treated area and a thick tissue slice was prepared. The white arrow indicates the approximate direction of injection of the saline including the drug capsules. The approximate scale is shown by the double arrow representing a length of about 200 microns. The result indicates homogeneous capsules distribution at a tissue depth of about 1 milimeter.

The devices disclosed herein can deliver any injectable material or agent either encapsulated or not. For example, there is delivery of Botulinum Toxin A: Botox® (Allergan, Irvine, Calif.) or Dysport® (Ipsen, Paris, FR) or any other Botulinum Toxin for the treatment of Over-Active Bladder or any other disorder in the bladder, to the bladder wall. Injection of Botulinum Toxin to the bladder wall using the devices disclosed herein will enable safe injection into predetermined depth together with homogenous drug dispersal in the treated area. It should be noted that any other material can be injected through the apparatus of the present invention without limiting the scope of the present invention.

The methods, devices and systems disclosed herein and illustrated in the drawings proposed are based on the use of a minimally invasive device, which delivers the injectable agent in such a manner that the treatment solution, containing either soluble agent or encapsulated agent suspension, is injected at close range through the bladder wall epithelium into the tissue beneath and is effectively delivered to the tissue at a pre-calibrated range of tissue depths.

It should be clear that the description of the embodiments and attached Figures set forth in this specification serves only for a better understanding of the invention, without limiting its scope as covered by the following claims. It should also be clear that a person skilled in the art, after reading the present specification can make adjustments or amendments to the attached Figures and above described embodiments that would still be covered by the following claims.

We claim:

1. A device for delivering substance to tissue comprising:
a delivery tube having proximal and distal ends;
a chamber in fluid connection with said delivery tube for containing the substance to be delivered to the tissue;
a plurality of needles having tips in fluid communication with said chamber;
a plunger coupled to a handle at the proximal end of said delivery tube, said plunger capable of being positioned within said delivery tube and movable relative thereto for delivering the substance through said needles; and
a protector having at least one orifice therein, such that when said device is in a retracted position the tips of said needles are on a first side of a distal end of said protector, and when said device is in an extended position the tips of said needles protrude through the at least one orifice to a second side of the distal end of said protector, the second side being opposite the first side.

2. The device of claim 1, further comprising a sheath attached at a distal end thereof to said protector, wherein said delivery tube is insertable through said sheath and movable therein between said retracted and extended positions.

3. The device of claim 1, wherein said protector is attached to said delivery tube and capable of being axially displaced therefrom between said retracted position and said extended position.

4. The device of claim 3, wherein said protector is attached to said delivery tube by a spring element biased so as to maintain said needles in the first position.

5. The device of claim 2, wherein said protector has one orifice having a diameter smaller than an inner diameter of the distal end of said sheath and capable of accommodating said plurality of needles in said extended position, said protector limiting extension of said plurality of needles in said extended position.

6. The device of claim 1, further comprising a plurality of cleaning pins respectively aligned with said plurality of needles for clearing inside bores thereof.

7. The device of claim 1, wherein said protector includes a plurality of orifices associated with said plurality of needles.

8. The device of claim 7, wherein at least one orifice of said plurality of orifices is set at an angle with respect to a center axis.

9. The device of claim 8, wherein a center orifice in said protector is aligned with said center axis, and wherein angles of said plurality of orifices increase as distance from the center orifice increases.

10. The device of claim 7, wherein said protector includes a plurality of orifices respectively associated with each of said plurality of needles, such that when in the extended position, the needles extend through said orifices at a plurality of angles relative to a center axis of said delivery tube.

11. The device of claim 10, wherein the tips of said plurality of needles have non-uniform height from a common base.

12. The device of claim 10, wherein a center needle has greatest height from a common base for all of said plurality of needles.

13. The device of claim 12, wherein a center orifice in said protector is aligned with said center axis, and wherein angles of said plurality of orifices from said center axis increase as distance from the center orifice increases.

14. The device of claim 1, wherein the tips of said plurality of needles have non-uniform height from a common base.

15. The device of claim 14, wherein a center needle has greatest height from a common base for all of said plurality of needles.

16. The device of claim 15, wherein said protector has a plurality of orifices respectively associated with said plurality of needles, and wherein a center orifice in said protector is aligned with said center axis, and wherein angles of said plurality of orifices increase as distance from the center orifice increases.

17. The device of claim 1, wherein at least a first needle is fixed relative to said catheter and at least one second needle is axially movable relative to said catheter.

18. The device of claim 17, wherein said at least one second needle is coupled to said catheter by a spring.

19. The device of claim 2, wherein said protector is adjustably fixed with respect to said sheath to vary distance of the tips of said needles from the distal end of said sheath when in said extended position.

20. The device of claim 1, further comprising an extension sensor adapted to activate an extension alert when said needles are extended to a predetermined distance from the retracted position.

21. The device of claim 1, wherein said delivery tube is sufficiently small in diameter to fit into an endoscopic device.

22. The device of claim 21, wherein said endoscopic device is selected from the group consisting of a resectoscope, a endoscope, a cytoscope, a enteroscope, and a colonoscope.

23. The device of claim 1, wherein said chamber is located at a proximal end of said delivery tube.

24. The device of claim 23, wherein each of said plurality of needles is associated with a respective delivery tube in fluid connection with said chamber, and further comprising an inner sheath binding together said plurality of delivery tubes.

25. The device of claim 1 further comprising a sheath attached at a distal end thereof to said protector, wherein said plurality of delivery tubes are insertable through said sheath and movable therein between said retracted and extended positions, and wherein at least a distal portion of said sheath is semi-rigid.

26. The device of claim 25, wherein said semi-rigid distal portion of said sheath is coupled to a rigid portion of said sheath.

27. The device of claim 2, further comprising a touch sensor adapted to activate a touch alert when said sheath is in contact with said tissue.

28. The device of claim 27, wherein said touch alert is a visual alert.

29. The device of claim 27, wherein said touch alert is an audible alert.

30. The device of claim 2, further comprising an extension sensor adapted to activate an extension alert when said needles are extended to a predetermined distance from the retracted position.

31. The device of claim 30, wherein said extension alert is a visual alert.

32. The device of claim 30, wherein said extension alert is an audible alert.

33. The device of claim 1, further comprising a fixing mechanism to lock said plurality of needles in the extended position.

34. The device of claim 1, further comprising an automatic substance dispensing module.

35. The device of claim 1, further comprising an automated substance injection module.

36. The device of claim 1, further comprising an anti-dripping valve associated with said delivery tube.
